(19) United States
(12) Patent Application Publication
(10) Pub. No.: US 2026/0232668 A1
(43) Pub. Date: Aug. 13, 2026

(54) Combination Therapy Comprising BTK Inhibitor and Belumosudil

(71) Applicant: Kadmon Corporation, LLC, Morristown, NJ (US)

(72) Inventors: Masha Poyurovsky, Cambridge, MA (US); Haya Taitel, Cambridge, MA (US)

(73) Assignee: Kadmon Corporation, LLC, Morristown, NJ (US)

(21) Appl. No.: 19/499,959

(22) PCT Filed: Jul. 17, 2024
(86) PCT No.: PCT/US2024/038315

§ 371 Date: Jan. 7, 2026

Related U.S. Application Data
(60) Provisional application No. 63/513,952, filed on Jul. 17, 2023.

(30) Foreign Application Priority Data
Dec. 5, 2023 (EP) 23214297.6

(51) Int. Cl. A61K 31/517; A61K 31/519; A61P 37/06

(52) U.S. Cl. CPC A61K 31/517; A61K 31/519; A61P 37/06

(57) ABSTRACT

Disclosed herein are methods of treating a disease selected from graft-versus-host disease (GVHD), systemic sclerosis (scleroderma), chronic lung allograft dysfunction (CLAD), restrictive allograft syndrome (RAS), and bronchiolitis obliterans syndrome (BOS) using a combination of a BTK inhibitor (such as rilzabrutinib) and belumosudil.

### CROSS-REFERENCE TO RELATED APPLICATIONS

This application claims priority to U.S. Provisional Application No. 63/513,952, filed on Jul. 17, 2023, and European Application No. 23214297.6, filed on Dec. 5, 2023, the disclosures of each of which are incorporated herein by reference in their entireties for any purpose.

### FIELD

The present disclosure relates to methods of treating a disease selected from graft-versus-host disease (GVHD), systemic sclerosis (scleroderma), chronic lung allograft dysfunction (CLAD), restrictive allograft syndrome (RAS), and bronchiolitis obliterans syndrome (BOS) using a combination of a BTK inhibitor (such as rilzabrutinib) and belumosudil.

### BACKGROUND

Bruton's tyrosine kinase (BTK) is a cytoplasmatic non-receptor tyrosine kinase belonging to the Tec family of kinases. BTK functions downstream of the B-Cell Receptor (BCR) and is essential to B cell lineage maturation and functional antibody production, and inhibition of BTK activity in cells produces phenotypic changes consistent with the blockade of the BCR. BTK inhibition results in the downregulation of various B-cell activities, including cell proliferation, differentiation, maturation, and survival, and the up-regulation of apoptosis. As such, BTK inhibitors have been investigated as therapeutics for treating a variety of cancers. However, BTK also plays a crucial role in signaling pathways that relate to autoimmune and immune-mediated diseases. For example, BTK inhibition may suppress production of autoantibodies thought to be important in the development of certain autoimmune diseases. In addition, BTK plays a role in the activation of innate immune cells, such as macrophages and neutrophils, which are key players in inflammation. Accordingly, a BTK inhibitor has the potential to target multiple pathways involved in inflammation and autoimmunity.

Belumosudil is an oral selective Rho-Associated Coiled-coil-containing protein Kinase-2 (ROCK2) inhibitor. ROCK2 inhibition acts on the dysregulated adaptive immune system and the fibrosis that occurs because of aberrant tissue repair. Studies have shown that belumosudil down-regulates proinflammatory responses and also inhibits aberrant pro-fibrotic signaling. The FDA approved belumosudil (REZUROCK®) in 2021 for the treatment of cGVHD after failure of at least two prior lines of systemic therapy.

Combination therapy is an attractive option for disease treatment, particularly using therapeutic agents that target distinct pathways. Accordingly, provided herein are methods of treating diseases such as graft-versus-host disease (GVHD), systemic sclerosis (scleroderma), chronic lung allograft dysfunction (CLAD), restrictive allograft syndrome (RAS), and bronchiolitis obliterans syndrome (BOS) using a combination of a BTK inhibitor and belumosudil.

### SUMMARY

Described herein are methods of treating a disease selected from graft-versus-host disease (GVHD), systemic sclerosis (scleroderma), chronic lung allograft dysfunction (CLAD), restrictive allograft syndrome (RAS), and bronchiolitis obliterans syndrome (BOS) using a combination of a BTK inhibitor and belumosudil.

Exemplary embodiments include the following.

Embodiment 1. A method of treating a disease or disorder selected from systemic sclerosis and a transplant-associated dysfunction in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 2. A method of treating a disease or disorder selected from graft-versus-host disease (GVHD), systemic sclerosis (scleroderma), chronic lung allograft dysfunction (CLAD), restrictive allograft syndrome (RAS), and bronchiolitis obliterans syndrome (BOS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 3. A method for treating graft-versus-host disease (GVHD) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 4. A method for treating systemic sclerosis (scleroderma) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 5. A method for treating chronic lung allograft dysfunction (CLAD) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 6. A method for treating restrictive allograft syndrome (RAS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 7. A method for treating bronchiolitis obliterans syndrome (BOS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

Embodiment 8. The method of any one of embodiments 1-7, wherein the BTK inhibitor is a reversible BTK inhibitor.

Embodiment 9. The method of any one of embodiments 1-7, wherein the BTK inhibitor is an irreversible BTK inhibitor.

Embodiment 10. The method of any one of embodiments 1-9, wherein the BTK inhibitor is (i) (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof; (ii) 1-[(3R)-3-[4-amino-3-(4-phenoxyphenyl)-1H-pyrazolo[3,4-d]pyrimidin-1-yl]-1-piperidinyl]-2-propen-1-one or a pharmaceutically acceptable salt thereof; or (iii) (4-amino-3-(4-phenoxyphenyl)-1-[(3R)-1-(prop-2-enoyl)piperidin-3-yl]-1,3-dihydro-2H-imidazo[4,5-c]pyridin-2-one) or a pharmaceutically acceptable salt thereof.

Embodiment 11. The method of embodiment 10, wherein the BTK inhibitor is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

Embodiment 12. A method for treating graft-versus-host disease (GVHD) in a human patient in need thereof comprising administering to the human patient a therapeutically effective amount of a combination comprising: (a) (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof, and (b) 2-{3-[4-(1H-indazol-5-ylamino)-2-quinazolinyl]phenoxy}-N-(propan-2-yl) acetamide or a pharmaceutically acceptable salt thereof.

Embodiment 13. The method of any one of embodiments 2, 3, and 12, wherein GVHD is chronic GVHD (cGVHD).

Embodiment 14. The method of any one of embodiments 1-13, wherein 2-{3-[4-(1H-indazol-5-ylamino)-2-quinazolinyl]phenoxy}-N-(propan-2-yl) acetamide or a pharmaceutically acceptable salt thereof is administered to the human patient at a daily dosage of up to about 400 mg.

Embodiment 15. The method of embodiment 14, wherein 2-{3-[4-(1H-indazol-5-ylamino)-2-quinazolinyl]phenoxy}-N-(propan-2-yl) acetamide, or a pharmaceutically acceptable salt thereof, is administered to the human patient at a daily dosage of about 50-400 mg.

Embodiment 16. The method of embodiment 14 or 15, wherein 2-{3-[4-(1H-indazol-5-ylamino)-2-quinazolinyl]phenoxy}-N-(propan-2-yl) acetamide, or a pharmaceutically acceptable salt thereof, is administered to the human patient at a dose of about 50 mg, 100 mg, 150 mg, or 200 mg.

Embodiment 17. The method of embodiment 16, wherein the dose is administered to the human patient once or twice daily.

Embodiment 18. The method of any one of embodiments 1-17, wherein 2-{3-[4-(1H-indazol-5-ylamino)-2-quinazolinyl]phenoxy}-N-(propan-2-yl) acetamide, or a pharmaceutically acceptable salt thereof, is administered orally.

Embodiment 19. The method of any one of embodiments 10-18, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a daily dosage of up to about 800 mg.

Embodiment 20. The method of embodiment 19, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a daily dosage of about 50-800 mg.

Embodiment 21. The method of embodiment 19 or 20, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a dose of about 100 mg, 200 mg, or 400 mg.

Embodiment 22. The method of embodiment 21, wherein the dose of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof is administered to the human patient once a day or twice a day.

Embodiment 23. The method of any one of embodiments 10-22, wherein the (E) isomer (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

Embodiment 24. The method of any one of embodiments 10-22, wherein the (Z) isomer (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

Embodiment 25. The method of any one of embodiments 10-22, wherein a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

Embodiment 26. The method of any one of embodiments 10-25, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered orally.

Embodiment 27. The method of any one of embodiments 10-26, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered separately.

Embodiment 28. The method of embodiment 27, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered sequentially.

Embodiment 29. The method of any one of embodiments 10-28, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered simultaneously.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the flow cytometry analysis of the B-cell functional assay described in Example 1. FIG. 1A shows activated B cells using anti-CD79b stimulation alone. FIG. 1B shows activated B cells following anti-CD79b stimulation and treatment with 10 μM rilzabrutinib, and FIG. 1C shows activated B cells following anti-CD79b stimulation and treatment with 10 μM belumosudil.

FIG. 2 shows a dose-response graph of belumosudil and rilzabrutinib of the anti-CD79b-mediated B-cell activation assay described in Example 1.

FIG. 3A shows average GVHD scores in the mouse model of GVHD measured daily up to Day 56, following therapeutic administration of rilzabrutinib and ibrutinib at the indicated doses, starting on Day 21 after disease initiation (dosing initiation is indicated by the vertical dotted line), as described in Example 2. FIG. 3B shows the total GVHD burden in the vehicle, rilzabrutinib, and ibrutinib treatment groups, of the same sclerodermatous GVHD model, represented as the AUC of each group's daily scores, as described in Example 2.

FIG. 4 shows animal survival as monitored on a daily basis for the duration of the study described in Example 4.

FIG. 5A shows the body weight change of animals treated in the study described in Example 4. Animals were weighed daily and body weight change as compared to Day 0 was calculated. The AUC was calculated from Day 0 until Day 56 using the trapezoidal rule transformation and is shown on the right. Statistical significance between groups was determined by one-way ANOVA with Dunnett's multiple comparisons test to compare all groups to GVHD vehicle-control group. **p<0.01. Data is presented as mean±SEM. n=6-12 per group.

FIG. 5B shows the body weight change—death weight carried of animals treated in the study described in Example 4. Animals were weighed daily and body weight change as compared to Day 0 was calculated. Data is shown with body weight with which an animal was found dead or was euthanized carried forward for the duration of the study. The AUC was calculated from Day 0 until Day 56 using the trapezoidal rule transformation and is shown on the right. Statistical significance between groups was determined by oneway ANOVA with Dunnett's multiple comparisons test to compare all groups to GVHD vehicle-control group. *p<0.05. Data is presented as mean±SEM. n=6-12 per group.

FIG. 6A shows the GVHD score—standard scoring of animals treated in the study described in Example 4. Animals were scored daily according to the protocol shown in Table 4. The AUC was calculated from Day 0 until study conclusion on Day 56 using the trapezoidal rule transformation and is shown on the right. Statistical significance was determined by Kruskal-Wallis with Dunn's post-hoc test to compare all groups to GVHD vehicle-control group. Data is presented as mean±SEM. **p<0.01. n=6-12 per group.

FIG. 6B shows the GVHD score—standard scoring—death score carried of animals treated in the study described in Example 4. Animals were scored daily according to the protocol shown in Table 4. Data is shown with GVHD scores with which an animal was found dead or was euthanized carried forward for the duration of the study. The AUC was calculated from Day 0 until study conclusion on Day 56 using the trapezoidal rule transformation and is shown on the right. Statistical significance was determined by Kruskal-Wallis with Dunn's post-hoc test to compare all groups to GVHD vehicle-control group. Data is presented as mean SEM. ***p<0.001. n=6-12 per group.

FIG. 7A shows the GVHD score—modified scoring of animals treated in the study described in Example 4. Animals were scored daily according to the protocol shown in Table 5. The AUC was calculated from Day 0 until study conclusion on Day 56 using the trapezoidal rule transformation to enable effective comparison of groups by statistical test. Statistical significance was determined by Kruskal-Wallis with Dunn's post-hoc test to compare all groups to the GVHD vehicle-control group. Data is presented as mean±SEM. **p<0.01. n=6-12 per group.

FIG. 7B shows the GVHD score—modified scoring—death score carried of animals treated in the study described in Example 4. Animals were scored daily according to the protocol shown in Table 5. The AUC was calculated from Day 0 until study conclusion on Day 56 to enable effective comparison of groups by statistical test. Statistical significance was determined by Kruskal-Wallis with Dunn's post-hoc test to compare all groups to the GVHD vehicle-control group. Data is presented as mean±SEM. ***p<0.001. n=6-12 per group.

FIG. 8A shows progression-free survival (standard GVHD scale) tracked for the duration of the study described in Example 4 and plotted as percent progression free survival.

FIG. 8B shows progression-free survival (modified scGVHD scale) tracked for the duration of the study described in Example 4 and plotted as percent progression free survival.

FIG. 9A shows the sum histopathology score for mouse lung of the animals treated in the study described in Example 4. Group mean±standard error of the mean (SEM). Cell transfers were associated with significant disease induction in the lungs of mice, with slight reductions in sum histopathology scores seen with either ibrunitib or rilzabrunitib treatment. Data analyzed by non-parametric one-way ANOVA with post-hoc Dunn's multiple comparisons tests. ** indicates p<0.01.

FIG. 9B shows the component histopathology score for mouse lung of the animals treated in the study described in Example 4. Group mean±standard error of the mean (SEM). Cell transfers were associated with significant disease induction in the lungs of mice, with slight reductions in sum histopathology scores seen with either ibrunitib or rilzabrunitib treatment. Data analyzed by non-parametric one-way ANOVA with post-hoc Dunn's multiple comparisons tests. ** indicates p<0.01.

FIG. 10A shows the sum histopathology score for mouse skin of the animals treated in the study described in Example 4. Group mean±SEM. Sum scores in the skin varied considerably, with a few samples per group exhibiting much more severe lesions compared to the group mean. Slightly lowered sum scores seen with ibrunitib and rilzabrunitib were associated with lowered hyperplasia and fibrosis histopathology scores (FIG. 10B). Data analyzed by non-parametric one-way ANOVA with post-hoc Dunn's multiple comparisons tests. ** indicates p<0.01.

FIG. 10B shows the histopathology scores for mouse skin of the animals treated in the study described in Example 4. Group mean±SEM. Data analyzed by non-parametric one-way ANOVA with post-hoc Dunn's multiple comparisons tests. ** indicates p<0.01.

FIG. 11 shows the dermal thickness measurement for mouse skin of the animals treated in the study described in Example 4. Group mean±SEM. Dermal thickness was measured in 5 sites and averaged per animal. Although the dermis was thicker in animals receiving cell transfers, the among-group statistical comparison was not significant. A slight reduction in dermal thickness corresponded with lowered dermal fibrosis scores (FIG. 10B) in animals treated with rilzabrunitib. Data analyzed by one-way ANOVA with post-hoc Tukey tests.

FIG. 12 shows animal survival as monitored on a daily basis for the duration of the study described in Example 5.

FIG. 13 shows the body weight change of animals treated in the study described in Example 5. Animals were weighed daily and body weight change as compared to Day 0 was calculated. The AUC was calculated using the trapezoidal rule transformation and is shown on the right. Statistical significance between groups was determined by one-way ANOVA with Tukey's multiple comparison post-test to compare all groups. ****p<0.0001. Data is presented as mean±SEM. n=6-12 per group.

FIG. 14 shows the body weight change—death weight carried of animals treated in the study described in Example 5. Animals were weighed daily and body weight change as compared to Day 0 was calculated. Data is shown with body weight with which an animal was found dead or was euthanized carried forward for the duration of the study. The AUC was calculated using the trapezoidal rule transformation and is shown on the right. Statistical significance between groups was determined by one-way ANOVA with Tukey's multiple comparison post-test to compare all groups. *p<0.05; ***p<0.001. Data is presented as mean±SEM. n=6-12 per group.

FIG. 15 shows the GVHD score—standard scoring of animals treated in the study described in Example 5. Animals were scored daily according to the protocol shown in Table 4. The AUC was calculated from Day 0 until study conclusion on Day 56 using the trapezoidal rule transformation and is shown on the right. Statistical significance was determined by one-way ANOVA with Tukey's multiple comparison post-test to compare all groups. Data is presented as mean±SEM. *p<0.05. n=6-12 per group.

FIG. 16 shows the GVHD score—standard scoring—death score carried of animals treated in the study described in Example 5. Animals were scored daily according to the protocol shown in Table 4. Data is shown with GVHD scores with which an animal was found dead or was euthanized carried forward for the duration of the study. The AUC was calculated from Day 0 until study conclusion on Day 56 using the trapezoidal rule transformation and is shown on the right. Statistical significance was determined by one-way ANOVA with Tukey's multiple comparison post-test to compare all groups. Data is presented as mean±SEM. n=6-12 per group.

FIG. 17 shows the GVHD score—modified scoring of animals treated in the study described in Example 5. Animals were scored daily according to the protocol shown in Table 5. The AUC was calculated from Day 0 until study conclusion on Day 56 using the trapezoidal rule transformation to enable effective comparison of groups by statistical test. Statistical significance was determined by one-way ANOVA with Tukey's multiple comparison post-test to compare all groups. Data is presented as mean±SEM. n=6-12 per group.

FIG. 18 shows the GVHD score—modified scoring—death score carried of animals treated in the study described in Example 5. Animals were scored daily according to the protocol shown in Table 5. The AUC was calculated from Day 0 until study conclusion on Day 56 to enable effective comparison of groups by statistical test. Statistical significance was determined by one-way ANOVA with Tukey's multiple comparison post-test to compare all groups. Data is presented as mean±SEM. n=6-12 per group.

FIG. 19 shows progression-free survival (standard GVHD scale) tracked for the duration of the study described in Example 5 and plotted as percent progression free survival.

FIG. 20 shows progression-free survival (modified scGVHD scale) tracked for the duration of the study described in Example 5 and plotted as percent progression free survival.

### DETAILED DESCRIPTION

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an”, and “the” include plural referents unless the context clearly dictates otherwise. In this application, the use of “or” means “and/or” unless stated otherwise. Furthermore, use of the term “including” as well as other forms, such as “include”, “includes”, and “included”, is not limiting.

Reference in the specification to “some embodiments”, “an embodiment”, “one embodiment”, or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. Accordingly, “about” includes the exact amount modified by the term as well as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range or a particular value. It is understood that use of the term “about” before a listing of numerical values or a range or values applies the term “about” to each of the listed numerical values or across the full range of values. For example, “about 50 mg, 100 mg, or 200 mg” is understood to mean the same as “about 50 mg, about 100 mg, or about 200 mg,” and “about 50-200 mg” is understood to mean the same as “about 50 mg to about 200 mg.”

“Administering” or “administered to” as used herein (for example, with reference to administration of a drug for treatment, such as belumosudil or rilzabrutinib, to a subject), refers to the act of prescribing medicine(s) containing the drug for the subject to take during treatment, the act of dispensing the medicine(s) to the subject, and/or the act of physically receiving or ingesting the medicine(s). Thus, the drug (for example, belumosudil or rilzabrutinib) can be “administered” by a physician or other medical professional who writes prescriptions for medicine(s); and/or by a pharmacist who fills said prescriptions and/or dispenses the medicine(s) to the subject; and/or by the patient or subject who ingests the medicine and/or his or her partner or caretaker.

As used herein, “belumosudil” refers to the compound having the chemical name 2-{3-[4-(1H-indazol-5-ylamino)-2-quinazolinyl]phenoxy}-N-(propan-2-yl) acetamide and the chemical structure shown below.

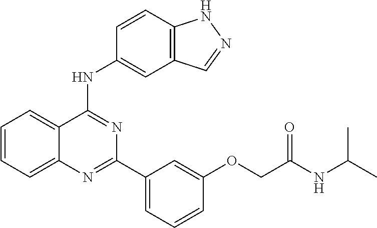 [SMILES: CC(C)NC(=O)COc1cccc(-c2nc(Nc3ccc4[nH]ncc4c3)c3ccccc3n2)c1]

Belumosudil is also known as KD025 and Slx-2119. Reference herein to “belumosudil” refers to the compound in any form, as well as pharmaceutically acceptable salts thereof, unless the context clearly indicates otherwise. The term “belumosudil” refers to the compound belumosudil (for example, in the free base form, amorphous form, and/or crystalline form), to pharmaceutically acceptable salts of belumosudil, for example, the mesylate salt form as used in as REZUROCK®, and to any form of belumosudil that may be used in a formulation or pharmaceutical composition for administering the compound to a patient. The mesylate salt of belumosudil is presently marketed in the United States and other countries under the tradename REZUROCK® (Kadmon Corp./Sanofi) for the treatment of patients with cGVHD, in some instances after failure of at least two prior lines of systemic therapy. The active pharmaceutical ingredient of REZUROCK® is belumosudil mesylate salt with the molecular formula C₂₇H₂₈N₆O₅S, a molecular weight of 548.62 g/mol, and the chemical name 2-{3-[4-(1H-indazol-5-ylamino)-2-quinazolinyl]phenoxy}-N-(propan-2-yl) acetamide methanesulfonate (1:1).

A “BTK inhibitor” or an “inhibitor of BTK” refers to a compound that inhibits Bruton's tyrosine kinase (BTK). An “irreversible BTK inhibitor” is characterized by a Michael acceptor moiety able to form a covalent bond with the conserved Cys481 residue in the ATP binding site. A “reversible BTK inhibitor” binds to a specific pocket in the SH3 domain through weak, reversible interactions (such as hydrogen bonds or hydrophobic interactions), causing an inactive conformation of the enzyme. In some embodiments, the BTK inhibitor is a hybrid BTK inhibitor in which the inhibitor binds to BTK in a reversible covalent manner, forming reversible covalent bonds with the Cys481 residue and temporarily inactivating the enzyme. An overview of BTK inhibitors is provided in Tasso et al., Molecules, 2021, 26, 7411, the disclosure of which is incorporated herein by reference in its entirety.

As used herein, “rilzabrutinib” refers to the compound having the chemical name (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and the chemical structure shown below.

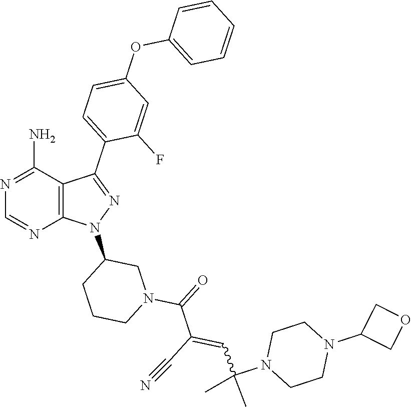 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

Rilzabrutinib is also known as PRN1008. This compound has been disclosed in several patent publications, such as, e.g., PCT Publication Nos. WO 2014/039899, WO 2015/127310, WO 2016/100914, WO 2016/105531, and WO 2018/005849, the contents of each of which are incorporated herein by reference in their entirety. Reference herein to “rilzabrutinib” refers to the compound in any form, as well as pharmaceutically acceptable salts thereof, unless the context clearly indicates otherwise. The term “rilzabrutinib” refers to the compound rilzabrutinib (for example, in the free base form, amorphous form, and/or crystalline form), to pharmaceutically acceptable salts of rilzabrutinib, and to any form of rilzabrutinib that may be used in a formulation or pharmaceutical composition for administering the compound to a patient.

“Pharmaceutically acceptable salts” refers to non-toxic, inorganic and organic acid addition salts of a compound, such as belumosudil or rilzabrutinib. In one embodiment, the pharmaceutically acceptable salt of belumosudil is the mesylate salt.

As used herein, a “dose” refers to a specified amount of medication, such as a compound described herein (for example, belumosudil or rilzabrutinib) taken at one time. For example, a dose of 200 mg of belumosudil refers to administering 200 mg of belumosudil to a subject at one time, for example as a tablet or capsule. The “dosage” refers to a specific amount, number, and frequency of doses over a specific period of time. For example, a daily dosage of 400 mg of belumosudil refers to administering 400 mg of belumosudil to a subject, as either a single dose or as multiple doses, over the course of one day (for example, belumosudil administered at a dose of 200 mg twice daily).

As used herein, “therapeutically effective amount” refers to the amount of a drug, such as belumosudil or rilzabrutinib, that provides an intended therapeutic effect. A “therapeutically effective amount” of a drug (such as belumosudil or rilzabrutinib) means an amount which, when administered to a human for treating a disease (for example, GVHD) is sufficient to effect treatment for the disease state being treated. As applied to any one of the diseases disclosed herein (i.e., GVHD, scleroderma, CLAD, RAS, or BOS), “treating” or “treatment” includes (1) reducing the risk of developing the disease (GVHD, scleroderma, CLAD, RAS, or BOS) and/or inhibiting the disease, i.e., arresting or reducing the development of the disease or its clinical symptoms; and (2) relieving the disease (GVHD, scleroderma, CLAD, RAS, or BOS), i.e., causing regression, reversal, or amelioration of the disease or reducing the number, frequency, duration, or severity of its clinical symptoms. The therapeutically effective amount of a drug may vary depending upon the health and physical condition of the subject to be treated, the extent of disease progression, the assessment of the medical situation, and other relevant factors.

As used herein, the terms “individual(s)”, “subject(s)”, and “patient(s)” mean any mammal and may be used interchangeably. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly, or a hospice worker). In some embodiments, the patient is a human patient.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Belumosudil

The methods of treating a disease selected from graft-versus-host disease (GVHD), systemic sclerosis (scleroderma), chronic lung allograft dysfunction (CLAD), restrictive allograft syndrome (RAS), and bronchiolitis obliterans syndrome (BOS) described herein include administration of belumosudil.

Belumosudil is a ROCK2 inhibitor. Belumosudil binds to and inhibits the serine/threonine kinase activity of ROCK2 and to a lesser extent ROCK1. Belumosudil inhibits ROCK2 and ROCK1 with IC₅₀ values of approximately 100 nM and 3 μM, respectively. As such, belumosudil is useful in treating diseases, disorders, and conditions regulated by ROCK including autoimmune and fibrotic disorders. Belumosudil has been approved by the FDA for the treatment of chronic graft-versus-host disease (cGVHD).

Belumosudil has the chemical name 2-{3-[4-(1H-indazol-5-ylamino)-2-quinazolinyl]phenoxy}-N-(propan-2-yl) acetamide and is represented by the chemical structure shown below.

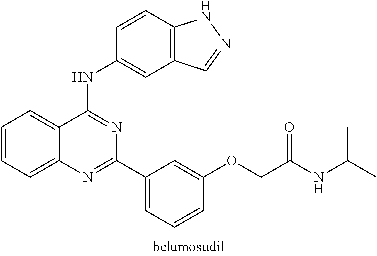 [SMILES: CC(C)NC(=O)COc1cccc(-c2nc(Nc3ccc4[nH]ncc4c3)c3ccccc3n2)c1]

In some embodiments, belumosudil is provided as a pharmaceutically acceptable salt. In some embodiments, belumosudil is provided as the mesylate salt. In some embodiments, belumosudil is provided as the free compound (i.e., not as a pharmaceutically acceptable salt).

In some embodiments, belumosudil is provided as a capsule or tablet for oral administration. In some embodiments, belumosudil is provided as a liquid formulation for oral administration.

In some embodiments, belumosudil is provided in a dose of from about 50 mg to about 400 mg belumosudil measured as the equivalent amount of free base. In some embodiments, belumosudil is provided in a dose of about 50 mg, 100 mg, 200 mg, or 400 mg. In some embodiments, belumosudil is provided in a dose of about 50 mg. In some embodiments, belumosudil is provided in a dose of about 100 mg. In some embodiments, belumosudil is provided in a dose of about 200 mg. In some embodiments, belumosudil is provided in a dose of about 400 mg.

In some embodiments, belumosudil is provided as a capsule or tablet. In some embodiments, belumosudil is provided as a capsule or tablet comprising about 200 mg of belumosudil. In some embodiments, belumosudil is provided as a tablet comprising 200 mg of belumosudil.

In some embodiments, belumosudil is provided as a liquid formulation comprising about 50 mg, 100 mg, 200 mg, or 400 mg of belumosudil. In some embodiments, belumosudil is provided as a liquid formulation comprising about 50 mg of belumosudil. In some embodiments, belumosudil is provided as a liquid formulation comprising about 100 mg of belumosudil. In some embodiments, belumosudil is provided as a liquid formulation comprising about 200 mg of belumosudil. In some embodiments, belumosudil is provided as a liquid formulation comprising about 400 mg of belumosudil.

### BTK Inhibitors

The methods of treating a disease selected from graft-versus-host disease (GVHD), systemic sclerosis (scleroderma), chronic lung allograft dysfunction (CLAD), restrictive allograft syndrome (RAS), and bronchiolitis obliterans syndrome (BOS) described herein include administration of a BTK inhibitor (also referred to herein as “an inhibitor of BTK”).

In some embodiments, the BTK inhibitor is a reversible inhibitor of BTK. In some embodiments, the BTK inhibitor is an irreversible inhibitor of BTK. In some embodiments, the BTK inhibitor is a hybrid BTK inhibitor in which the inhibitor binds to BTK in a reversible covalent manner. In some embodiments, the BTK inhibitor is a BTK inhibitor described in Tasso et al., Molecules, 2021, 26, 7411, the disclosure of which is incorporated herein by reference.

In some embodiments, the BTK inhibitor is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof (rilzabrutinib). In some embodiments, the BTK inhibitor is 1-[(3R)-3-[4-amino-3-(4-phenoxyphenyl)-1H-pyrazolo[3,4-d]pyrimidin-1-yl]-1-piperidinyl]-2-propen-1-one or a pharmaceutically acceptable salt thereof (ibrutinib). In some embodiments, the BTK inhibitor is (4-amino-3-(4-phenoxyphenyl)-1-[(3R)-1-(prop-2-enoyl)piperidin-3-yl]-1,3-dihydro-2H-imidazo[4,5-c]pyridin-2-one) or a pharmaceutically acceptable salt thereof (tolebrutinib).

In some embodiments, the BTK inhibitor is fenebrutinib. In some embodiments, the BTK inhibitor is evobrutinib. In some embodiments, the BTK inhibitor orelabrutinib. In some embodiments, the BTK inhibitor remibrutinib. In some embodiments, the BTK inhibitor BIIB-091. In some embodiments, the BTK inhibitor tirabrutinib. In some embodiments, the BTK inhibitor acalabrutinib. In some embodiments, the BTK inhibitor vecabrutinib. In some embodiments, the BTK inhibitor zanubrutinib. In some embodiments, the BTK inhibitor poseltinib. In some embodiments, the BTK inhibitor pirtobrutinib. In some embodiments, the BTK inhibitor spebrutinib. In some embodiments, the BTK inhibitor olmutinib. In some embodiments, the BTK inhibitor branebrutinib. In some embodiments, the BTK inhibitor TAK-020. In some embodiments, the BTK inhibitor elsubrutinib. In some embodiments, the BTK inhibitor is tolebrutinib.

In some embodiments, the BTK inhibitor is provided in Table 1.

TABLE 1
Selected BTK Inhibitors and Chemical Structures.

| BTK | |
|---|---|
| Inhibitor | Structure |
| rilzabrutinib | 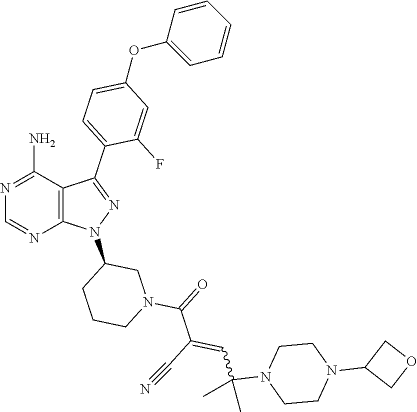 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1] |
| tolebrutinib | 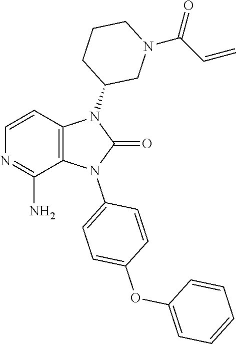 [SMILES: C=CC(=O)N1CCC[C@@H](n2c(=O)n(-c3ccc(Oc4ccccc4)cc3)c3c(N)nccc32)C1] |
| evobrutinib | 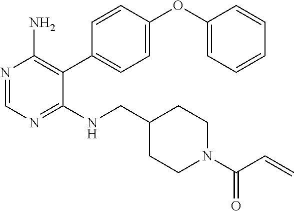 [SMILES: C=CC(=O)N1CCC(CNc2ncnc(N)c2-c2ccc(Oc3ccccc3)cc2)CC1] |
| remibrutinib | 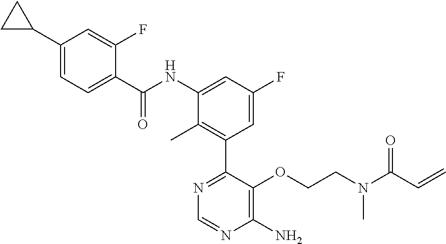 [SMILES: C=CC(=O)N(C)CCOc1c(N)ncnc1-c1cc(F)cc(NC(=O)c2ccc(C3CC3)cc2F)c1C] |
| tirabrutinib | 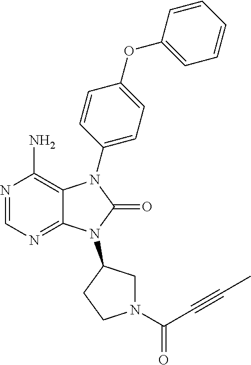 [SMILES: CC#CC(=O)N1CC[C@@H](n2c(=O)n(-c3ccc(Oc4ccccc4)cc3)c3c(N)ncnc32)C1] |
| vecabrutinib | 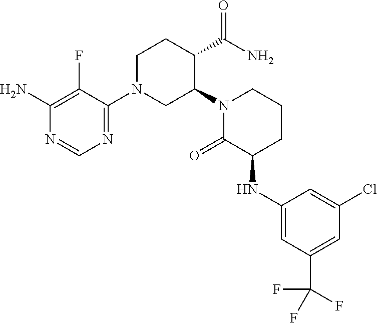 [SMILES: NC(=O)[C@H]1CCN(c2ncnc(N)c2F)C[C@@H]1N1CCC[C@@H](Nc2cc(Cl)cc(C(F)(F)F)c2)C1=O] |
| poseltinib | 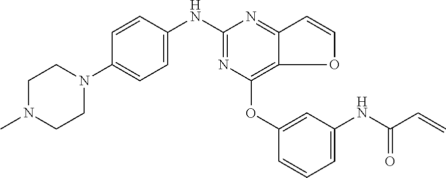 [SMILES: C=CC(=O)Nc1cccc(Oc2nc(Nc3ccc(N4CCN(C)CC4)cc3)nc3ccoc23)c1] |
| spebrutinib | 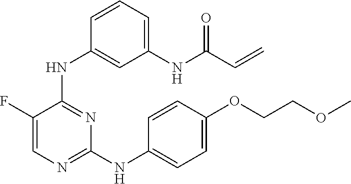 [SMILES: C=CC(=O)Nc1cccc(Nc2nc(Nc3ccc(OCCOC)cc3)ncc2F)c1] |
| branebrutinib | 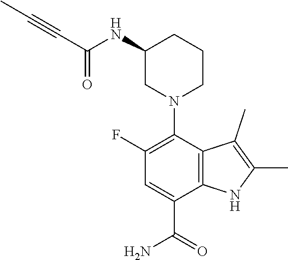 [SMILES: CC#CC(=O)N[C@H]1CCCN(c2c(F)cc(C(N)=O)c3[nH]c(C)c(C)c23)C1] |
| elsubrutinib | 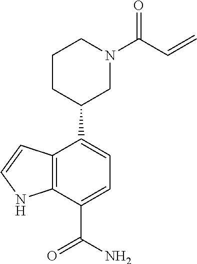 [SMILES: C=CC(=O)N1CCC[C@@H](c2ccc(C(N)=O)c3[nH]ccc23)C1] |
| ibrutinib | 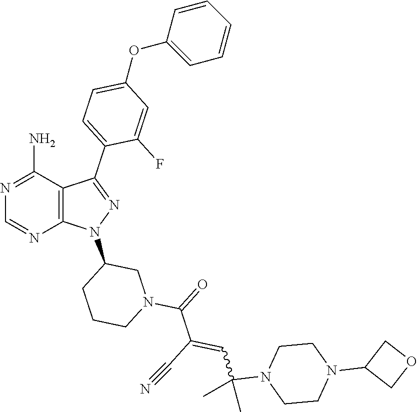 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1] |
| fenebrutinib | 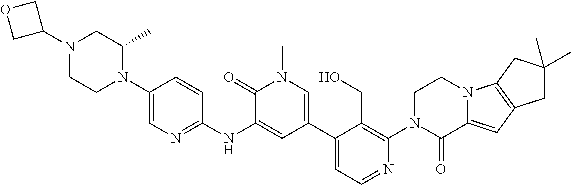 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1] |
| orelabrutinib | 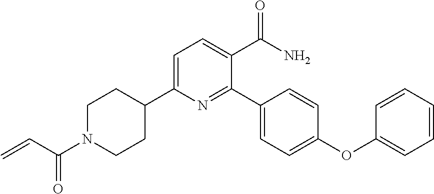 [SMILES: C=CC(=O)N1CCC(c2ccc(C(N)=O)c(-c3ccc(Oc4ccccc4)cc3)n2)CC1] |
| BIIB-091 | 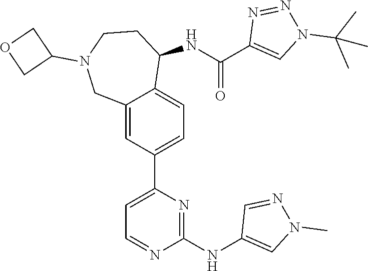 [SMILES: Cn1cc(Nc2nccc(-c3ccc4c(c3)CN(C3COC3)CC[C@H]4NC(=O)c3cn(C(C)(C)C)nn3)n2)cn1] |
| acalabrutinib | 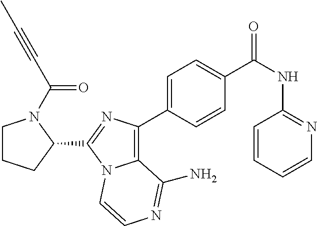 [SMILES: CC#CC(=O)N1CCC[C@H]1c1nc(-c2ccc(C(=O)Nc3ccccn3)cc2)c2c(N)nccn12] |
| zanubrutinib | 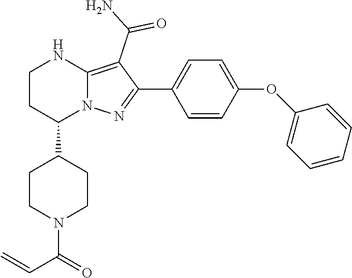 [SMILES: C=CC(=O)N1CCC([C@@H]2CCNc3c(C(N)=O)c(-c4ccc(Oc5ccccc5)cc4)nn32)CC1] |
| pirtobrutinib | 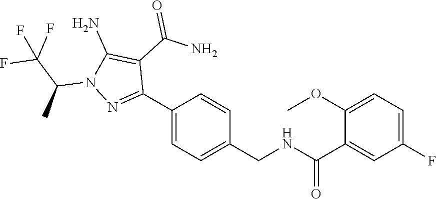 [SMILES: COc1ccc(F)cc1C(=O)NCc1ccc(-c2nn([C@@H](C)C(F)(F)F)c(N)c2C(N)=O)cc1] |
| olmutinib | 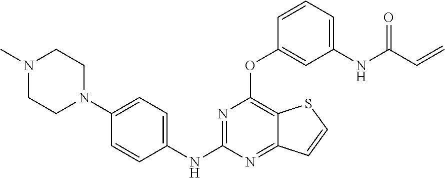 [SMILES: C=CC(=O)Nc1cccc(Oc2nc(Nc3ccc(N4CCN(C)CC4)cc3)nc3ccsc23)c1] |
| TAK-020 | 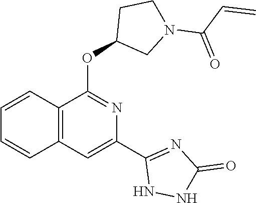 [SMILES: C=CC(=O)N1CC[C@H](Oc2nc(-c3nc(=O)[nH][nH]3)cc3ccccc23)C1] |

### Rilzabrutinib

In some embodiments, the BIK inhibitor used in the methods described herein is rilzabrutinib.

Rilzabrutinib is a highly selective BTK inhibitor. Rilzabrutinib functions as a reversible covalent BTK inhibitor and forms both a non-covalent and a covalent bond with its target, allowing for enhanced selectivity and extended inhibition with low systemic exposure. Rilzabrutinib's reversible binding minimizes the likelihood of permanently modified peptides. Rilzabrutinib is currently being developed for the treatment of immune-mediated diseases.

Rilzabrutinib has the chemical name (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and is represented by the chemical structure shown below.

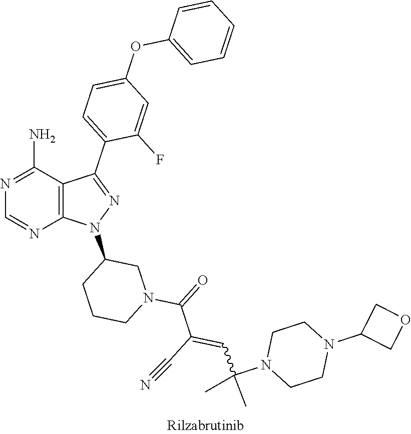 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

Rilzabrutinib is also known as 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-pent-2-enenitrile; 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-a-[2-methyl-2-[4-(3-oxetanyl)-1-piperazinyl]propylidene]-p-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonproprietary-names-(inn)/pl121.pdf9sfvrsn=69617906_15&download=true) having the following structure:

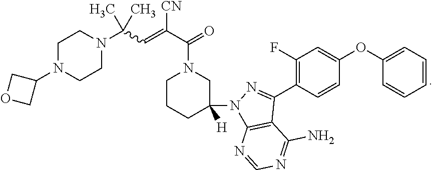 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

Rilzabrutinib exists as either the (Z) isomer or the (E) isomer. A dose of the (E) isomer of rilzabrutinib may contain the corresponding (Z) isomer as an impurity in less than about 2% by weight, such as less than about 1% by weight; a dose of the (Z) isomer of rilzabrutinib may contain the corresponding (E) isomer as an impurity in less than about 2% by weight, such as less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 2% by weight. In some embodiments, the molar ratio of (E) to (Z) isomer is 8:2. In some embodiments, the molar ratio of (E) to (Z) isomer is 9:1. In some embodiments, rilzabrutinib is provided as the (E) isomer. In some embodiments, rilzabrutinib is provided as the (Z) isomer. In some embodiments, rilzabrutinib is provided as a mixture of (E) and (Z) isomers.

In some embodiments, rilzabrutinib is provided as a pharmaceutically acceptable salt. In some embodiments, rilzabrutinib is provided as the free base (i.e., not as a pharmaceutically acceptable salt).

In some embodiments, rilzabrutinib is formulated for oral administration. In some embodiments, rilzabrutinib is provided as a capsule or tablet for oral administration. In some embodiments, rilzabrutinib is provided as a liquid formulation for oral administration.

In some embodiments, rilzabrutinib is provided in a dose of from about 50 mg to about 800 mg rilzabrutinib. In some embodiments, rilzabrutinib is provided in a dose of about 50 mg, 75 mg, 100 mg, 200 mg, 300 mg, or 400 mg. In some embodiments, rilzabrutinib is provided in a dose of about 100 mg, 200 mg, or 400 mg. In some embodiments, rilzabrutinib is provided in a dose of about 100 mg. In some embodiments, rilzabrutinib is provided in a dose of about 200 mg. In some embodiments, rilzabrutinib is provided in a dose of about 400 mg.

For oral administration, rilzabrutinib can be provided in the form of tablets containing about 1 to about 1000 mg of the active ingredient, particularly about 1, 5, 10, 15, 20, 25, 50, 75, 100, 200, 300, 400, 500, 600, 750, and 800 mg of the active ingredient. In some embodiments, rilzabrutinib is provided as a capsule or tablet comprising about 100 mg or 300 mg of rilzabrutinib. In some embodiments, rilzabrutinib is provided as a capsule or tablet comprising about 100 mg of rilzabrutinib. In some embodiments, rilzabrutinib is provided as a capsule or tablet comprising about 300 mg of rilzabrutinib.

In some embodiments, rilzabrutinib is provided as a liquid formulation comprising about 50 mg, 150 mg, 300 mg, 300 mg BID, 450 mg BID, or 600 mg, of rilzabrutinib. In some embodiments, rilzabrutinib is provided as a liquid formulation comprising about 300 mg of rilzabrutinib. In some embodiments, rilzabrutinib is provided as a liquid formulation comprising about 300 mg BID of rilzabrutinib. In some embodiments, rilzabrutinib is provided as a liquid formulation comprising about 450 mg BID of rilzabrutinib. In some embodiments, rilzabrutinib is provided as a liquid formulation comprising about 600 mg of rilzabrutinib.

### Methods of Treatment

In one aspect, provided herein are methods of treating a disease or disorder selected from systemic sclerosis and a transplant-associated dysfunction in a human patient in need thereof comprising administering to the human patient a therapeutically effective amount of a combination comprising a BTK inhibitor and belumosudil. In some embodiments, the disease or disorder is systemic sclerosis. In some embodiments, the disease or disorder is a transplant-associated dysfunction. In some embodiments, the transplant-associated dysfunction is graft-versus-host disease (GVHD), chronic lung allograft dysfunction (CLAD), restrictive allograft syndrome (RAS), or bronchiolitis obliterans syndrome (BOS). In some embodiments, the transplant-associated dysfunction is GVHD. In some embodiments, the transplant-associated dysfunction is CLAD. In some embodiments, the transplant-associated dysfunction is RAS. In some embodiments, the transplant-associated dysfunction is BOS.

In a further aspect, provided herein are methods of treating a disease or disorder selected from graft-versus-host disease (GVHD), systemic sclerosis (scleroderma), chronic lung allograft dysfunction (CLAD), restrictive allograft syndrome (RAS), and bronchiolitis obliterans syndrome (BOS) in a human patient in need thereof comprising administering to the human patient a therapeutically effective amount of a combination comprising a BTK inhibitor and belumosudil. Also provided herein are methods of treating GVHD in a human patient in need thereof comprising administering to the human patient a therapeutically effective amount of a combination comprising a BTK inhibitor and belumosudil. Further, provided herein are methods of treating CLAD in a human patient in need thereof comprising administering to the human patient a therapeutically effective amount of a combination comprising a BTK inhibitor and belumosudil. Also provided herein are methods of treating RAS in a human patient in need thereof comprising administering to the human patient a therapeutically effective amount of a combination comprising a BTK inhibitor and belumosudil. Also provided herein are methods of treating BOS in a human patient in need thereof comprising administering to the human patient a therapeutically effective amount of a combination comprising a BTK inhibitor and belumosudil. Also provided herein are methods of treating systemic sclerosis (scleroderma) in a human patient in need thereof comprising administering to the human patient a therapeutically effective amount of a combination comprising a BTK inhibitor and belumosudil.

Also provided herein is a combination of a BTK inhibitor and belumosudil for use in a method of treating a disease or disorder selected from systemic sclerosis and a transplant-associated dysfunction in a human patient in need thereof. Further provided herein is use of a combination of a BTK inhibitor and belumosudil for the manufacture of a medicament for treating a disease or disorder selected from systemic sclerosis and a transplant-associated dysfunction in a human patient in need thereof.

Also provided herein is a combination of a BTK inhibitor and belumosudil for use in a method of treating a disease or disorder selected from GVHD, scleroderma, CLAD, RAS, and BOS in a human patient in need thereof. Further provided herein is use of a combination of a BTK inhibitor and belumosudil for the manufacture of a medicament for treating a disease or disorder selected from GVHD, scleroderma, CLAD, RAS, and BOS in a human patient in need thereof.

In some embodiments, the disease or disorder is graft-versus-host disease (GVHD). In some embodiments, the GVHD is chronic GVDH (cGVHD). In some embodiments, the GVHD is sclerodermatous chronic GVHD. In some embodiments, the GVHD is lung GVHD. In some embodiments, the GVHD is lung cGVHD.

In some embodiments, the disease or disorder is systemic sclerosis (scleroderma).

In some embodiments, the disease or disorder is chronic lung allograft dysfunction (CLAD).

In some embodiments, the disease or disorder is restrictive allograft syndrome (RAS).

In some embodiments, the disease or disorder is bronchiolitis obliterans syndrome after lung transplant (BOS).

In some embodiments, the disease or disorder is a transplant-associated dysfunction.

The diagnosis of any of the diseases or disorders disclosed herein can be made by a qualified healthcare worker, such as a medical doctor, based on clinically accepted criteria.

### Subject

In some embodiments, the subject has graft-versus-host disease (GVHD). In some embodiments, the subject has chronic graft-versus-host disease (cGVHD).

In some embodiments, the subject has systemic sclerosis (scleroderma).

In some embodiments, the subject has chronic lung allograft dysfunction (CLAD).

In some embodiments, the subject has restrictive allograft syndrome (RAS).

In some embodiments, the subject has bronchiolitis obliterans syndrome after lung transplant (BOS).

In some embodiments, the subject previously received an organ transplant. In some embodiments, the subject is a bone marrow transplant recipient. In some embodiments, the subject is a lung transplant recipient. In some embodiments, the subject is a single lung transplant recipient. In some embodiments, the subject is a double lung transplant recipient.

In some embodiments, the subject is an adult. In some embodiments, the adult is a male. In other embodiments, the adult is a female. In some embodiments, the adult is at least age 20, 25, 30, 35, 40, 45, 50, 55, 60, 65, 70, 75, 80, 85, 90, or 95 years of age. In some embodiments, the subject is a child or adolescent. In some embodiments, the subject is at least 12 years of age. In some embodiments, the subject is at least 12, 13, 14, 15, 16, 17, 18, or 19 years of age.

### Administration

In some embodiments, belumosudil is administered orally to the subject. In some embodiments, the BTK inhibitor is administered orally to the subject. In some embodiments, rilzabrutinib is administered orally to the subject. In some embodiments, the combination comprising a BTK inhibitor and belumosudil is administered orally to the subject. In some embodiments, the combination comprising rilzabrutinib and belumosudil is administered orally to the subject.

In some embodiments, the combinations disclosed herein (e.g., belumosudil and a BTK inhibitor) are administered to the subject with food.

In some embodiments, the BTK inhibitor and belumosudil are administered separately to the subject. In some embodiments, the BTK inhibitor and belumosudil are administered sequentially to the subject. In some embodiments, belumosudil is administered to the subject prior to the administration to the subject of the BTK inhibitor. In some embodiments, the BTK inhibitor is administered to the subject prior to the administration to the subject of belumosudil. In some embodiments, the BTK inhibitor and belumosudil are administered simultaneously to the subject. In some embodiments, the BTK inhibitor and belumosudil are administered to the subject on the same day. In some embodiments, the BTK inhibitor and belumosudil are administered to the subject with timing such that both the BTK inhibitor and belumosudil are active (i.e., not completely metabolized) in the subject. In some embodiments, the BTK inhibitor and belumosudil are administered to the subject on the same day and within 6 hours of administering both agents, such as within 1 hour, 2 hours, 3 hours, 4 hours, 5 hours, or 6 hours. In some embodiments, the BTK inhibitor and belumosudil are administered to the subject on different days. In some embodiments, the BTK inhibitor and belumosudil are administered to the subject on different days and within 3 weeks of administering both agents, such as within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

In some embodiments, rilzabrutinib and belumosudil are administered separately to the subject. In some embodiments, rilzabrutinib and belumosudil are administered sequentially to the subject. In some embodiments, belumosudil is administered to the subject prior to the administration to the subject of rilzabrutinib. In some embodiments, rilzabrutinib is administered to the subject prior to the administration to the subject of belumosudil. In some embodiments, rilzabrutinib and belumosudil are administered simultaneously to the subject. In some embodiments, rilzabrutinib and belumosudil are administered to the subject on the same day. In some embodiments, rilzabrutinib and belumosudil are administered to the subject on different days.

In some embodiments, belumosudil is administered to the subject once or twice daily. In some embodiments, belumosudil is administered to the subject once per day. In some embodiments, belumosudil is administered to the subject twice per day.

In some embodiments, the BTK inhibitor is administered to the subject once a day, twice a day, or three times a day. In some embodiments, the BTK inhibitor is administered to the subject once a day. In some embodiments, the BTK inhibitor is administered to the subject twice a day. In some embodiments, the BTK inhibitor is administered to the subject three times a day.

In some embodiments, rilzabrutinib is administered to the subject once a day or twice a day. In some embodiments, rilzabrutinib is administered to the subject once a day. In some embodiments, rilzabrutinib is administered to the subject twice a day.

In some embodiments, the combination treatment (i.e., the BTK inhibitor and belumosudil) is continued based on the patient's tolerability until active disease (GVHD, scleroderma, CLAD, RAS, or BOS) in symptoms resolve or progress. In some embodiments, the belumosudil treatment is continued based on the patient's tolerability until active disease symptoms resolve or progress. In some embodiments, the BTK inhibitor (such as rilzabrutinib) treatment is continued based on the patient's tolerability until active disease symptoms resolve or progress.

The duration of the treatment is patient-dependent. In some embodiments, the duration of the treatment is 1 month, 2 months, 3 months, 6 months, 9 months, 12 months, 15 months, 18 months, 21 months or 24 months. In some embodiments, the duration of treatment is further extended by up to another 24 months or longer until disease progression.

In some embodiments, the methods of treating a disease described herein (such as GVHD, scleroderma, CLAD, RAS, or BOS) provide a method of improving the FEV₁ in a subject with the disease (such as GVHD, scleroderma, CLAD, RAS, or BOS).

In some embodiments, the methods disclosed herein reduce the risk of an organ (such as a lung) re-transplantation in a subject.

In some embodiments, the methods disclosed herein improve the quality of life in a subject that has a disease or disorder selected from graft-versus-host disease (GVHD), systemic sclerosis (scleroderma), chronic lung allograft dysfunction (CLAD), restrictive allograft syndrome (RAS), and bronchiolitis obliterans syndrome (BOS).

In some embodiments, the methods disclosed herein reduce death, reduce progressive bronchiolar ectasia, reduce organ failure, reduce decline in lung function, increase recovery and stabilization post organ transplantation, decrease hospitalization, decrease health care utilization, and/or reduce the risk of re-transplantation.

In some embodiments, the methods disclosed herein comprise administration of belumosudil, as part of the combination described herein, at a dose that is lower than belumosudil administered as a single agent (i.e., monotherapy). In some embodiments, the methods disclosed herein comprise administration of belumosudil, as part of the combination described herein, at a daily dosage that is lower than belumosudil administered as a single agent (i.e., monotherapy). As such, in some instances, undesired side effects caused by administration of belumosudil may be diminished.

In some embodiments, the methods disclosed herein comprise administration of a BTK inhibitor (such as rilzabrutinib), as part of the combination described herein, at a dose that is lower than the BTK inhibitor administered as a single agent (i.e., monotherapy). In some embodiments, the methods disclosed herein comprise administration of a BTK inhibitor (such as rilzabrutinib), as part of the combination described herein, at a daily dosage that is lower than the BTK inhibitor administered as a single agent (i.e., monotherapy). As such, in some instances, undesired side effects caused by administration of the BTK inhibitor may be diminished.

In some embodiments, the methods disclosed herein comprising administration of a combination comprising belumosudil and a BTK inhibitor (such as rilzabrutinib) are more efficacious than administration of either belumosudil or BTK inhibitor alone (i.e., monotherapy).

### Dosing of Belumosudil

In any of the methods disclosed herein belumosudil is administered to the human patient at a daily dosage of up to about 400 mg measured as the equivalent amount of free base. In some embodiments, belumosudil is administered at a daily dosage of about 50 mg, 100 mg, 200 mg, or 400 mg. In some embodiments, belumosudil is administered at a daily dosage of about 50 mg. In some embodiments, belumosudil is administered at a daily dosage of about 100 mg. In some embodiments, belumosudil is administered at a daily dosage of about 200 mg. In some embodiments, belumosudil is administered at a daily dosage of about 400 mg.

In some embodiments, belumosudil is administered at a dose of about 50 mg, 100 mg, 200 mg, or 400 mg. In some embodiments, belumosudil is administered at a dose of about 50 mg. In some embodiments, belumosudil is administered at a dose of about 100 mg. In some embodiments, belumosudil is administered at a dose of about 200 mg. In some embodiments, belumosudil is administered at a dose of about 400 mg.

In some instances, the dose and/or daily dosage of belumosudil may need to be adjusted. For example, coadministration of belumosudil with strong CYP3A inducers (inducers of the CYP3A family of P-450 isoenzymes including CYP3A4) decreases belumosudil exposure, which may reduce the efficacy of belumosudil. In such cases, the dose and/or daily dosage of belumosudil should be increased. In another example, coadministration of belumosudil with proton pump inhibitors decreases belumosudil exposure, which may reduce the efficacy of belumosudil. In such cases, the dose and/or daily dosage of belumosudil should be increased.

In some embodiments, belumosudil is administered to the human patient once or twice daily. In some embodiments, belumosudil is administered to the human patient once daily. In some embodiments, belumosudil is administered to the human patient twice daily.

In some embodiments, belumosudil is administered to the human patient orally. In some embodiments, belumosudil is administered to the human patient as a capsule or tablet. In some embodiments, belumosudil is administered to the human patient as a liquid formulation.

### Dosing of Rilzabrutinib

In any of the methods disclosed herein, rilzabrutinib is administered to the human patient at a daily dosage of up to about 800 mg. In some embodiments, rilzabrutinib is administered at a daily dosage of about 100 mg, 300 mg, 400 mg, 600 mg, or 800 mg.

In some embodiments, rilzabrutinib is administered to the human patient at a dose of about 50 mg, 75 mg, 100 mg, 200 mg, 300 mg, or 400 mg. In some embodiments, rilzabrutinib is administered to the human patient at a dose of about 100 mg, 200 mg, or 400 mg. In some embodiments, rilzabrutinib is administered to the human patient at a dose of about 100 mg. In some embodiments, rilzabrutinib is administered to the human patient at a dose of about 200 mg. In some embodiments, rilzabrutinib is administered to the human patient at a dose of about 400 mg.

In some embodiments, rilzabrutinib is administered to the human patient once a day or twice a day. In some embodiments, rilzabrutinib is administered to the human patient once a day. In some embodiments, rilzabrutinib is administered to the human patient twice a day.

In some embodiments, rilzabrutinib is administered to the human patient orally. In some embodiments, rilzabrutinib is administered to the human patient as a capsule or tablet. In some embodiments, rilzabrutinib is administered to the human patient as a liquid formulation.

### Dosing of Ibrutinib

In any of the methods disclosed herein ibrutinib is administered to the human patient at a daily dosage of up to about 420 mg. In some embodiments, ibrutinib is administered at a daily dosage of from about 140 mg to about 420 mg, such as from about 150 mg to about 400 mg, from about 150 mg to about 350 mg, from about 150 mg to about 300 mg, from about 150 mg to about 250 mg, from about 150 mg to about 200 mg, from about 200 mg to about 400 mg, from about 200 mg to about 350 mg, from about 200 mg to about 300 mg, from about 200 mg to about 250 mg, from about 250 mg to about 400 mg, from about 250 mg to about 350 mg, from about 250 mg to about 300 mg, from about 300 mg to about 400 mg, and from about 350 to about 400 mg. In some embodiments, ibrutinib is administered at a daily dosage of about 140 mg, 150 mg, 200 mg, 250 mg, 300 mg, 350 mg, 400 mg, or 420 mg.

In some embodiments, ibrutinib is administered at a daily dosage of about 140 mg. In some embodiments, ibrutinib is administered at a daily dosage of about 150 mg. In some embodiments, ibrutinib is administered at a daily dosage of about 200 mg. In some embodiments, ibrutinib is administered at a daily dosage of about 250 mg. In some embodiments, ibrutinib is administered at a daily dosage of about 300 mg. In some embodiments, ibrutinib is administered at a daily dosage of about 350 mg. In some embodiments, ibrutinib is administered at a daily dosage of about 400 mg. In some embodiments, ibrutinib is administered at a daily dosage of about 420 mg.

In some embodiments, ibrutinib is administered to the human patient at a dose of from about 140 mg to about 420 mg, such as from about 150 mg to about 400 mg, from about 150 mg to about 350 mg, from about 150 mg to about 300 mg, from about 150 mg to about 250 mg, from about 150 mg to about 200 mg, from about 200 mg to about 400 mg, from about 200 mg to about 350 mg, from about 200 mg to about 300 mg, from about 200 mg to about 250 mg, from about 250 mg to about 400 mg, from about 250 mg to about 350 mg, from about 250 mg to about 300 mg, from about 300 mg to about 400 mg, and from about 350 to about 400 mg. In some embodiments, ibrutinib is administered at a dose of about 140 mg, 150 mg, 200 mg, 250 mg, 300 mg, 350 mg, 400 mg, or 420 mg. In some embodiments, ibrutinib is administered to the human patient at a dose of about 140 mg. In some embodiments, ibrutinib is administered to the human patient at a dose of about 200 mg. In some embodiments, ibrutinib is administered to the human patient at a dose of about 250 mg. In some embodiments, ibrutinib is administered to the human patient at a dose of about 300 mg. In some embodiments, ibrutinib is administered to the human patient at a dose of about 400 mg. In some embodiments, ibrutinib is administered to the human patient at a dose of about 420 mg.

In some embodiments, ibrutinib is administered to the human patient once a day, twice a day, or three times a day. In some embodiments, ibrutinib is administered to the human patient once a day. In some embodiments, ibrutinib is administered to the human patient twice a day. In some embodiments, ibrutinib is administered to the human patient three times a day.

In some embodiments, ibrutinib is administered to the human patient orally. In some embodiments, ibrutinib is administered to the human patient as a capsule or tablet. In some embodiments, ibrutinib is administered to the human patient as a liquid formulation.

### Dosing of Tolebrutinib

In any of the methods disclosed herein, tolebrutinib is administered to the human patient at a daily dosage of from about 60 mg to about 120 mg. In some embodiments, tolebrutinib is administered at a daily dosage of about 60 mg. In some embodiments, tolebrutinib is administered at a daily dosage of about 120 mg.

In some embodiments, tolebrutinib is administered to the human patient at a dose of from about 60 mg to about 120 mg. In some embodiments, tolebrutinib is administered to the human patient at a dose of about 60 mg. In some embodiments, tolebrutinib is administered to the human patient at a dose of about 120 mg.

In some embodiments, tolebrutinib is administered to the human patient once a day.

In some embodiments, tolebrutinib is administered to the human patient orally. In some embodiments, tolebrutinib is administered to the human patient as a capsule or tablet.

### Kits/Article of Manufacture

Disclosed herein, in certain embodiments, are kits and articles of manufacture for use with one or more of the methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic. In some embodiments, the kit comprises belumosudil and a BTK inhibitor. In some embodiments, the kit comprises belumosudil and rilzabrutinib. In some embodiments, the kit comprises rilzabrutinib.

A kit typically includes labels listing contents and/or instructions for use, and package inserts with instructions for use. A set of instructions will also typically be included. In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

Also disclosed herein are pharmaceutical compositions for use in the methods described herein. In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. In Vitro Study of Belumosudil and Rilzabrutinib on B-Cell Activation in Human Whole Blood

### Background and Objective.

Belumosudil functions in GVHD primarily through the downregulation of the effector T cells and STAT3 signaling (Flynn et al, Blood, 2016). The effects of belumosudil on B cells, also integral in the regulation of aberrant inflammation, are less well characterized. The BTK inhibitor rilzabrutinib is known to block BCR-driven B-cell activation. The objective of this study was to test the ability of belumosudil to block BCR-driven activation of B-cells from human whole blood in order to determine whether belumosudil utilizes the same signaling pathway as rilzabrutinib.

### Method.

To examine the role of belumosudil and rilzabrutinib in B-cell activation, 95 μL of human whole blood was aliquoted per well before the addition of various concentrations of belumosudil (ASTATECH, 42507) and rilzabrutinib (Sanofi) ranging from 10 μM to 0.00017 μM in a 3-fold dilution series in PBS (Gibco, 20012-027). The whole blood was incubated at 37° C., 5% CO₂ for 1 h with belumosudil or rilzabrutinib prior to the addition of 80 ng/mL anti-CD79b antibody (BD Biosciences, 557592, clone: 3A2-2E7) to activate B cells overnight at 37° C., 5% CO₂. The next day, the whole blood was lysed with BD PharmLyse (BD Biosciences, 555899) and stained with FITC Mouse Anti-Human CD20 Antibody (BD Biosciences, 555622, clone: 2H₇), APC Mouse Anti-Human CD69 Antibody (BD Biosciences, 555533, clone: FN50) and Fixable Viability Dye eFluor 780 (eBioscience, 65-0865-14) in Stain Buffer (0.5% BSA (Gibco, 15260-037) and 2 mM EDTA (Invitrogen, AM9261) in PBS). Flow cytometric analyses were conducted using BD LSRFortessa™ Cell Analyzer (BD Biosciences) and the data analyzed by FlowJo (BD Biosciences). B-cell activation is determined by an increase in surface expression of activation marker CD69 on B cells (CD20+) following anti-CD79b stimulation. The experiment was conducted in duplicate.

### Results.

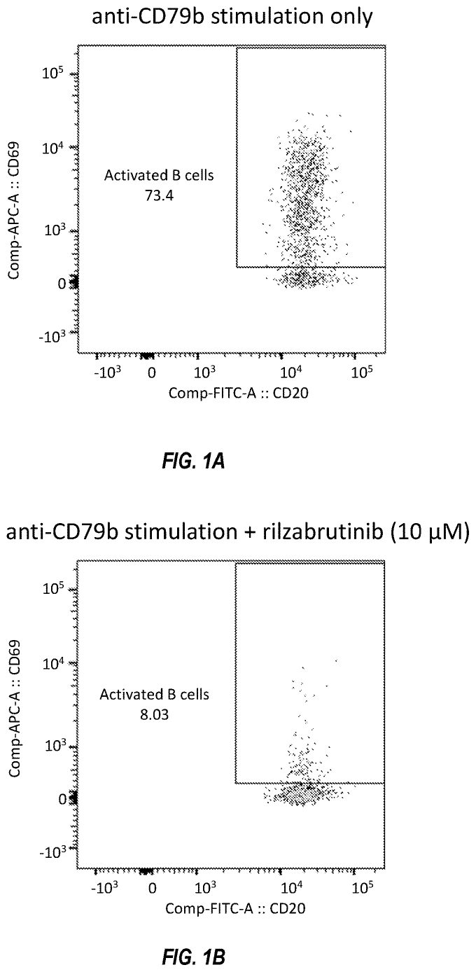

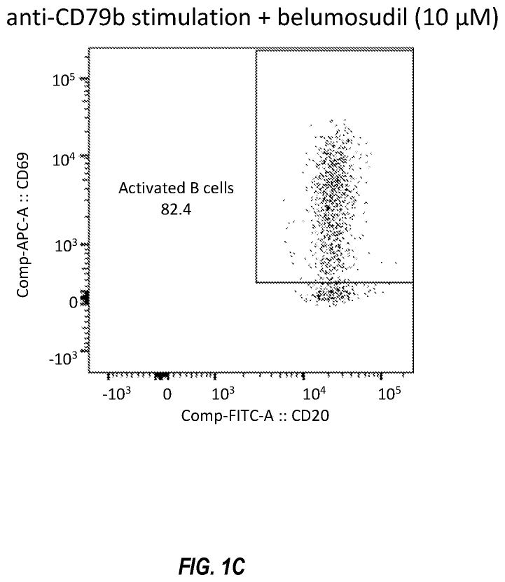

Consistent with the mechanism of action, flow cytometric analysis showed that 10 μM rilzabrutinib inhibited anti-CD79b-mediated B-cell activation in whole blood, decreasing the numbers of activated cells from 73.4% to 8.03% (FIGS. 1A-B). In contrast, flow cytometric analysis showed that belumosudil did not inhibit anti-CD79b-mediated B-cell activation in whole blood (FIG. 1C), with 82.4% B cells showing positive staining for CD69, a marker of activation, following treatment with 10 μM belumosudil.

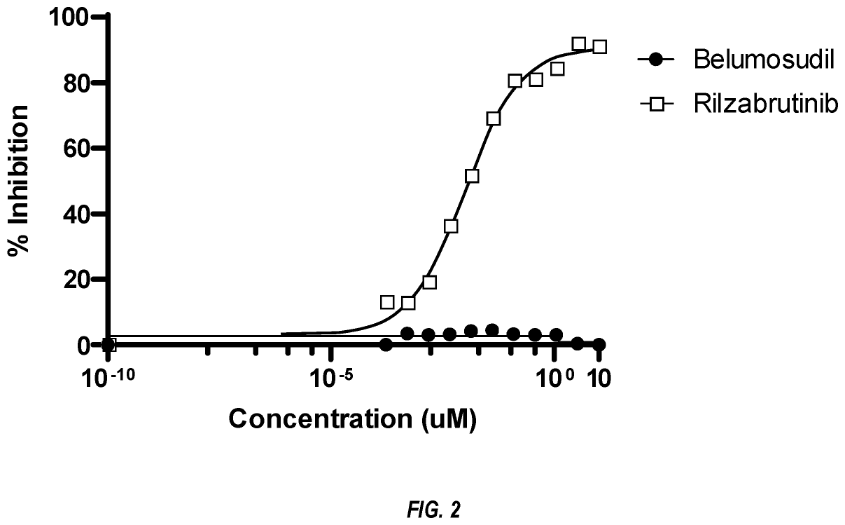

The dose-response graph of belumosudil and rilzabrutinib provided in FIG. 2 further demonstrates that belumosudil did not inhibit B-cell activation in whole blood, whereas rilzabrutinib inhibited B-cell activation in whole blood in a dose-dependent manner with IC₅₀ of 9.5 nM.

### Summary.

Rilzabrutinib inhibits B-cell activation, whereas no effect on B-cell activation was observed using various concentrations of belumosudil. The ROCK2 pathway (used by belumosudil) is dispensable downstream of BCR signaling, which differentiates it from the BTK pathway (used by rilzabrutinib). As such, belumosudil and rilzabrutinib utilize distinct signaling pathways, both of which have integral roles in the development of GVHD and CLAD. These data provide a mechanistic rationale for a therapeutic combination of ROCK and BTK targeting agents for the treatment of certain diseases by using a multi-pathway approach.

### Example 2. Efficacy of BTK Inhibitors Rilzabrutinib and Ibrutinib in a Mouse Model of Sclerodermatous Chronic Graft-Versus-Host Disease (cGVHD)

TABLE 2
Study Design (Single Agent).

| | | | | | | | Sacrifice & |
|---|---|---|---|---|---|---|---|
| | No. | TBI | Cell Transfer | | | | Collections |
| Group | Animals | Day −1 | Day 0 | Treatment | Schedule | Photo | Day 56 |
| 1 | 12 | 8.5 Gy | 4 × 10⁶ splenocytes | Vehicle | BID. PO | Days | 1. Blood |
| | | | 1 × 10⁷ bone marrow | | D21-55 | 33, 36, | (plasma) |
| 2 | 10 | 8.5 Gy | 4 × 10⁶ splenocytes | Ibrutinib | *BID, PO | 39, 42, 45 | 2. Lungs |
| | | | 1 × 10⁷ bone marrow | 25 mg/kg | D21-55 | | Left (fixed) |
| | | | | | | | Right |
| | | | | | | | (frozen) |
| 3 | 10 | 8.5 Gy | 4 × 10⁶ splenocytes | Rilzabrutinib | BID, PO | | 3. Skin |
| | | | 1 × 10⁷ bone marrow | 20 mg/kg | D21-55 | | 1 cm² (fixed) |
| | | | | | | | 1 cm² |
| | | | | | | | (frozen) |
| 4 | 10 | 8.5 Gy | 4 × 10⁶ splenocytes | Rilzabrutinib | BID. PO | | 4. Spleen |
| | | | 1 × 10⁷ bone marrow | 40 mg/kg | D21-55 | | frozen |

*Ibrutinib was administered for the AM dose, followed by 0.5% MC vehicle for the PM dose.

The effect of selective BTK inhibitors on disease progression was assessed in a sclerodermatous chronic GVHD model. Sclerodermatous GVHD was initiated by exposing female c57Bl/6 recipient mice to 8.5 Gy of total body irradiation on Day −1. Recipient: C57Bl/6 (CD45.1), (8 weeks old) and donor: LP/J (000676), (6-10 weeks old) female mice were obtained from Jackson Labs and allocated treatment and control groups. Animals were not replaced during the course of the study. Mice were handled in accordance with standard conventional procedures. Animals were acclimatized for a minimum of 3 days prior to study commencement. During this period, the animals were observed daily in order to reject animals that present in poor condition. The study was performed in animal rooms provided with filtered air at a temperature of 70±5° F. and 50% 20% relative humidity and an automatic timer for a light/dark cycle of 12 hours on and 12 hours off with no twilight.

On Day 0, a combination of bone marrow cells and splenocytes were transferred from donor LP/J mice to the recipients, as indicated in Table 2. Specifically, the C57Bl/6 (CD45.1) recipients were given an intravenous (IV) injection of a combination of splenocytes and bone marrow cells in sterile 1× PBS from donor female LP/J (CD45.2) mice. The spleen cells were isolated using the Miltenyi GentleMACS Dissociators. The BM cells were isolated using standard flushing practices. Post-transplant, all animals were housed under standard environmental conditions and maintained on the appropriate irradiated sterile rodent chow and sterile water ad libitum. The animals were dosed with vehicle, ibrutinib, or rilzabrutinib as indicated. All animals were monitored daily, and weight, survival, and disease progression were recorded following the scoring system described in Table 4.

On Day 20, animals were redistributed into groups so that the average scGVHD score was similar across all groups. Treatment was initiated on Day 21 and continued until Day 55. All dosing was performed BID to control for stress associated with oral gavage. Animals receiving ibrutinib were administered ibrutinib for the AM dose, followed by vehicle for the PM dose. On Days 33, 36, 39, 42, and 45, all animals were photographed under isoflurane anesthesia to assess disease severity.

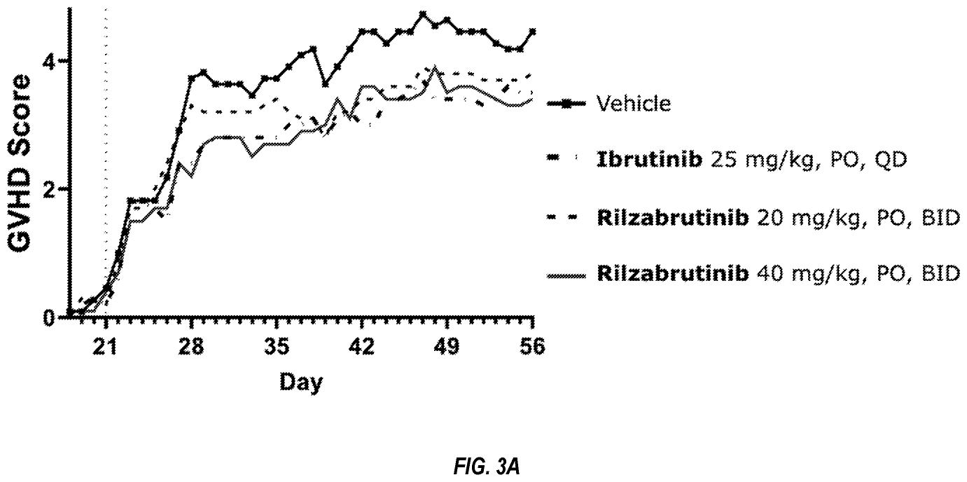

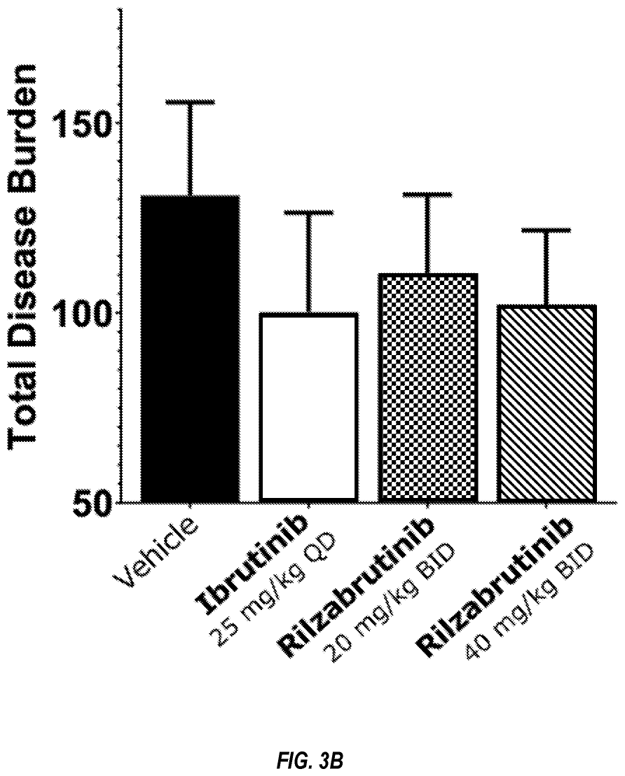

Treatment with ibrutinib at 25 mg/kg provided a numerical reduction in GVHD scores similar to what was previously reported in Dubovsky et al., JCI 2014, 124(11): 4867-4876 (See FIG. 2). Data relating to GVHD scores and total disease burden following treatment with ibrutinib and rilzabrutinib are shown in FIG. 3A and FIG. 3B. The effects of rilzabrutinib were dose-dependent, with a greater reduction in disease severity observed at the higher 40 mg/kg dose. The overall extent of disease reduction was similar to ibrutinib, which is approved for the treatment of steroid-refractory GVHD in adult humans.

These results demonstrate that BTK inhibitors can improve disease pathology in a GVHD model and, specifically, that rilzabrutinib, which has not been previously tested in this disease setting, has disease-modifying activity on par with ibrutinib, an approved agent for this indication. The results also establish a safe and effective dose of rilzabrutinib, which can be used in combination with belumosudil for studying the combination in animals.

### Example 3. Efficacy of BTK Inhibitor and Belumosudil Combination in Mouse Model of Sclerodermatous Chronic Graft-Versus-Host Disease (cGVHD)

This study will evaluate the effect of belumosudil and a BTK inhibitor on a murine model of sclerodermatous chronic GVHD.

### Materials.

The study will be performed under the same conditions as the single-agent study described in Example 2.

Mice will be treated with the test articles described below.

Vehicle only. The vehicle contains 0.5% methylcellulose (MC) (Sigma #M0262). The vehicle will be administered orally to the mice at a dose of 0.1 mL/20 g, twice daily on Day 21-Day 55.

Belumosudil. Belumosudil is formulated with vehicle (0.5% methylcellulose (MC)). Belumosudil will be administered orally to mice of Groups 4, 7, and 8 at a dose of 125 mg/kg as follows. Animals will be treated twice daily on Day 21-55.

Group 4. Belumosudil will be administered to animals for the first/AM dose only, and 0.5% MC vehicle will be administered for the last/PM dose.

Group 7. Belumosudil will be formulated in combination with ibrutinib for the first/AM dose and 0.5% MC vehicle will be administered for the last/PM dose. A test formulation for the combination will be performed prior to the study start.

Group 8. Belumosudil will be formulated in combination with rilzabrutinib for the first/AM dose, and 0.5% MC vehicle will be administered for the last/PM dose. A test formulation for the combination will be performed prior to the study start.

Ibrutinib. Ibrutinib (PCI-32765; Selleckchem, Cat. #S2680) is formulated with vehicle (0.5% methylcellulose (MC)). Ibrutinib will be administered orally to mice of Groups 5 and 7 at a dose of 20 mg/kg as follows. Animals will be treated twice daily on Day 21-55.

Group 5. Ibrutinib will be administered to animals for the first/AM dose only, and 0.5% MC vehicle will be administered for the last/PM dose.

Group 7. Ibrutinib will be formulated in combination with belumosudil for the first/AM dose, and 0.5% MC vehicle will be administered for the last/PM dose. A test formulation for the combination will be performed prior to the study start.

Rilzabrutinib. Rilzabrutinib is formulated with vehicle (0.5% methylcellulose (MC)). Rilzabrutinib will be administered orally to mice of Groups 6 and 8 at a dose of 40 mg/kg as follows. Animals will be treated twice daily on Day 21-55. Rilzabrutinib doses will be prepared as follows: weigh the required amount of rilzabrutinib for 3 days worth of dosing and add to mortar; add vehicle and grind with pestle for 5 minutes; transfer to a clear glass vial and sonicate for 30 minutes or until an even suspension is formed; dilute as needed for dosing solutions and prepare daily aliquots; and vortex/sonicate as needed prior to dosing.

Group 6. Rilzabrutinib will be administered to animals for the first/AM dose only, and 0.5% MC vehicle will be administered for the last/PM dose.

Group 8. Rilzabrutinib will be formulated in combination with belumosudil for the first/AM dose, and rilzabrutinib alone will be formulated for the last/PM dose. A test formulation for the combination will be performed prior to the study start.

### Experimental Design.

A summary of the study design is provided in Table 3.

TABLE 3
Study Design (Combination).

| | | | | | | | Sacrifice & |
|---|---|---|---|---|---|---|---|
| | No. | TBI | Cell Transfer | | | | Collections |
| Group | Animals | Day −1 | Day 0 | Treatment | Schedule | Photo | Day 56 |
| 1 | 6 | — | — | — | — | Days | 5. Blood |
| | | | | | | 33, 36, | (plasma) |
| 2 | 12 | 8.5 Gy | 4 × 10⁶ | Vehicle | *BID PO | 39, 42, 45 | 6. Lungs |
| | | | splenocytes | | D21-55 | | Left |
| 3 | 10 | | 1 × 10⁷ bone | Belumosudil | | | (fixed) |
| | | | marrow | **125 mg/kg | | | Right |
| 4 | 10 | | | Ibrutinib | | | (frozen) |
| | | | | 20 mg/kg | | | |
| 5 | 10 | | | Rilzabrutinib | | | 7. Skin |
| | | | | 40 mg/kg | | | 1 cm² |
| | | | | | | | (fixed) |
| 6 | 10 | | | Belumosudil | | | 1 cm² |
| | | | | **125 mg/kg | | | (frozen) |
| | | | | + | | | 4. Spleen |
| | | | | Ibrutinib | | | (frozen) |
| | | | | 20 mg/kg | | | |
| 7 | 10 | | | Belumosudil | | | |
| | | | | **125 mg/kg | | | |
| | | | | + | | | |
| | | | | Rilzabrutinib | | | |
| | | | | 40 mg/kg | | | |

*Belumosudil and/or ibrutinib administered for first/AM dose, followed by 0.5% MC vehicle for last/PM dose.
**Animals are dosed with 125 mg/kg belumosudil from Days 21-32. Animals in Groups 4, 7, and 8 are dosed with 100 mg/kg belumosudil from Days 33-55.

Mice will be randomized into one (1) group of six (6), one (1) group of twelve (12), and six (6) groups of ten (10) animals each upon arrival. Sclerodermatous chronic GVHD will be induced in C57Bl/6 (CD45.1) mice using a single acute dose of 8.5 Gy of total body irradiation (TBI) on Day −1.

All animals will be monitored on a daily basis to record weight change, survival, GVHD score (Table 4), and incidence of diarrhea and bloody stool. In addition, a second scoring system for sclerodermatous chronic GVHD (scGVHiD) will be used (Table 5).

TABLE 4
Standard GVHD Scoring Protocol.

| Criteria | Grade 0 | Grade 1 | Grade 2 |
|---|---|---|---|
| Weight loss | <10% | >10% < 25% | >25% |
| Posture | Normal | Hunching noted | Severe gait, impaired |
| | | only at rest | movement |
| Activity | Normal | Mild to moderately | Stationary until stimulated |
| | | decreased | |
| Fur texture | Normal | Mild to moderate | Severe ruffling and/or poor |
| | | ruffling | grooming |
| Skin | Normal | Scaling of paws | Obvious areas of denuded |
| integrity | | and/or tail | skin |

TABLE 5
Modified Scoring Protocol for scGVHD.

| Criteria | Grade 0 | Grade 1 | Grade 2 | Grade 3 | Grade 4 |
|---|---|---|---|---|---|
| Weight loss | no weight | >0% < 5% | >5% < 10% | >10% < 15% | >15% |
| | loss | | | | |
| Posture | Normal | Mild hunched | Moderate | Severe hunched | — |
| | | posture | hunched | posture | |
| | | | posture | | |
| Activity | Normal | Slowed gait | Slowed gait; | Immobility | — |
| | | | refusal to move | when touched | |
| | | | when touched | | |
| Fur texture | Normal | Ruffled hair; | Hair loss in a | Hair loss in a | Complete |
| | | small amount | single area <1 | single area >1 | hair loss or |
| | | of hair loss | cm² | cm² | >1 cm² area |
| | | | | | involved |
| Skin | Normal | Red/irritated | Skin | Scabbing or | Scabbing or |
| integrity | | skin lesion | flaking/ | bleeding; single | bleeding; |
| | | | peeling; | lesion | multiple |
| | | | single lesion | | lesions |

On Day 20, animals in Groups 3-8 will be redistributed into groups so that the average scGVHD score is similar across Groups 3-8. Redistribution will be based on the composite, 5-component scGVHD score described in Table 5.

Animals will be dosed with vehicle or test articles as detailed in Table 3. Dosing will start on Day 21 and continue until Day 55. Animals receiving ibrutinib will only be administered ibrutinib for the first/AM dose, followed by vehicle for the last/PM dose.

On Days 33, 36, 39, 42, and 45, all animals will be photographed under isoflurane anesthesia (top-down picture, plain white background) to assess disease severity. Each photo will be tagged with the corresponding animal's number to aid identification.

Animals that appear to be in pain will be administered Buprenorphine BID, as needed. Animals that are unable to right themselves, cold to the touch, or moribund will be euthanized. Animals requiring euthanasia will be euthanized by CO₂ inhalation and will not have collections performed. Animals that are found dead will not have collections performed.

On Day 56, all surviving animals will be sacrificed by CO₂ inhalation and organs detailed in Table 2 will be collected.

### Experimental Procedures.

GVH1D Induction. All recipient animals will receive a total body irradiation dose of 8.5 Gy on Day −1 as detailed in Table 2. On Day 0, Group 3-8 animals will receive a bone marrow (BM) transplant containing bone marrow cells in combination with splenocytes obtained from donor LP/J (CD45.2) mice via the tail vein (200 μL) according to Table 2. On Day 0, Group 2 animals will receive a bone marrow (BM) transplant containing only bone marrow cells obtained from donor LP/J (CD45.2) mice via the tail vein (200 μL) according to Table 2. The spleen cells will be isolated using the Miltenyi Gentlemacs Dissociators. The BM cells will be isolated using standard flushing practices (femur and tibia) and counted. Post-transplant, all recipient animals will be housed under standard environmental conditions and will be maintained on the appropriate rodent chow and sterile water ad libitum.

Supportive Care. Following TBI on Day −1 up to Day 8, all animals will be given supplemental high calorie highly palatable food in a dish on the bottom of the cage. Following transfer on Day 0, and then again as needed, all animals will be given 1 mL/animal/day of supplemental fluids (warmed ringers solution) via subcutaneous injection. An additional 1 mL fluid may be given in the pm on an as needed basis. Any animal exceeding 15% body weight loss will be given highly palatable soft food and 1 mL supplemental fluids (warmed ringers solution) via subcutaneous injection once per day. Any animal exceeding 20% body weight loss will be given 1 mL supplemental fluids (warmed ringers solution) via subcutaneous injection twice per day. Animals that appear to be in pain will be administered Buprenorphine BID, as needed.

Dosing. Animals in Group 1 will not be administered any treatments throughout the study. Animals in Groups 2-8 will be administered vehicle or test articles by oral gavage (PO) with dosing beginning on Day 21 and administered twice per day (BID), until Day 55 as follows. Animals in Groups 2, 3, and 6 will receive vehicle, or rilzabrutinib as indicated in Table 2 for both BID doses. Animals in Groups 4, 5 and 7 will be administered ibrutinib and/or belumosudil for the first/AM dose and vehicle for the last/PM dose each day of dosing. Animals in Group 8 will be administered rilzabrutinib and belumosudil for the first/AM dose and rilzabrutinib alone for the last/PM dose each day of dosing.

In-life Monitoring. Animals will be observed daily in order to assess possible differences among treatment groups and/or possible toxicity resulting from the treatments. Daily readouts will be weight change, survival, standard GVHD score (Table 3), modified GVHD score (Table 4), incidence of diarrhea, and incidence of bloody stool.

A clinical score for GVHD will be obtained daily for the entire study duration as assessed by a standard scoring system (Table 3) and modified scoring system (Table 4). The standard GVHD score is based on 5 criteria: percentage of weight change, posture (hunching), activity, fur texture, and skin integrity (maximum index=10). The modified GVHD score is based on 5 criteria: percentage of weight change, posture (hunching), activity, fur texture, and skin integrity (maximum index=18). The overall score will be reported, and the scores for each individual parameter will be recorded.

On Days 33, 36, 39, 42, and 45 all animals will be photographed under isoflurane anesthesia (top-down picture, plain white background) to assess disease severity. Each photo will be tagged with the corresponding animal's number to aid identification.

Animals Found Dead or Moribund. Animals that are unable to right themselves, cold to the touch, moribund, or in excess of 30% weight loss will be euthanized. Animals requiring euthanasia will be euthanized by CO₂ inhalation and will not have organs collected.

Sacrifice. On Day 56, all surviving animals will be sacrificed by xylazine overdose and organs detailed in Table 2 will be collected.

Sample Collection. Blood, lungs, skin, and spleens will be collected at sacrifice.

Blood. Prior to sacrifice, ~0.2 mL of blood will be collected from all animals by retro-orbital bleed into K₂EDTA tubes. The blood will be centrifuged, and the plasma collected and stored at −80° C.

Lung. The lungs will be removed and whole lung and right lung will be weighed. The right lung will be tied off on the right bronchus, excised below the tie, snap frozen in liquid nitrogen and stored at −80° C. The left lung will be insufflated with 10% NBF, tied off on the trachea to maintain insufflation, fixed in 10% NBF for 24 hours, then moved to PBS prior to processing for histopathology.

Skin. Prior to collection, the skin will be shaved. A 1×1 cm portion of the skin (from the back of the animal, between the shoulders, closer to head) will be excised, trimmed of any excess fat and/or connective tissue, sandwiched between foam in a tissue cassette, and placed in formalin. After 24 hours, the skin will be transferred to PBS for storage until subsequent histological analyses. A second 1 cm×1 cm portion of the skin (from the back of the animal, between the shoulders, just below the first piece) will be excised, trimmed of any excess fat and/or connective tissue, weighed, flash frozen, and stored at −80° C. If a visible lesion is present, it will be collected in a way that captures part of it in both fixed and frozen sections.

Spleen. The spleen will be excised and trimmed of any excess connective tissue. The spleen will then be weighed, flash frozen, and stored at −80° C.

Histopathology. Fixed lung and skin samples will be embedded in paraffin, sectioned at 5 microns, and slides stained with hematoxylin and eosin (H&E). One slide per block will be sectioned and stained with hematoxylin and eosin (H&E). All slides will be evaluated and scored with light microscopy by a board-certified veterinary pathologist.

### Example 4. Activity of BTK Inhibitors Rilzabrutinib and Ibrutinib in a Mouse Model of Sclerodermatous Chronic Graft-Versus-Host Disease (cGVHD)

The main objective of this study was to evaluate the effect of select BTK inhibitors on a murine model of sclerodermatous chronic graft-versus-host disease (GVHD).

### Animals.

Recipient female C57Bl/6 (CD45.1) mice (n=68; 8 weeks) with an average starting body weight (±SEM) of 18.75±0.12 g were obtained from Jackson Laboratories (Bar Harbor, Maine). Donor female LP/J mice (n=93; 6-10 weeks) were obtained from Jackson Laboratories (Bar Harbor, Maine). Animals were acclimatized for at least three days prior to study commencement. During this period, the animals were observed daily in order to reject any that presented in poor condition.

The study was performed in animal rooms provided with HEPA filtered air at a temperature of 70±5° F. and 50%±20% relative humidity. Animals were housed in groups of 6-10 per cage. Animal rooms were set to maintain a minimum of 12 to 15 air changes per hour. The room was on an automatic timer for a light/dark cycle of 12 hours on and 12 hours off with no twilight. Alpha-dri® bedding was used. Cages, tops, and water bottles were washed with a commercial detergent and allowed to air dry. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number, and treatment group was used to mark all cages. The temperature and relative humidity were recorded during the study, and the records retained.

Animals were fed with LabDiet 5053 rodent diet and water was provided ad libitum. Following total body irradiation (TBI) on Day −1 up to Day 8, all animals were given supplemental high-calorie highly palatable food.

Animals were randomized into seven (7) groups at the start of the study: one group of six (6) mice, one group of twelve (12), and five (5) groups of ten (10) mice each. Each animal was identified by an ear punch corresponding to an individual number. A cage card was used to identify each cage and was marked with the study number, treatment group number, and animal numbers.

### Study Design.

Sclerodermatous chronic GVHD was induced in C57Bl/6 (CD45.1) mice using a single acute dose of 8.5 Gy of TBI on Day −1. On Day 0, the C57Bl/6 (CD45.1) recipients were given an intravenous (IV) injection of a combination of splenocytes and bone marrow cells in sterile 1×PBS. Group 1 served as the naïve control group and did not receive either TBI or a cell transfer. Group 2 received an allogeneic cell transfer from donor LP/J mice consisting of only bone marrow cells obtained from donor female LP/J (CD45.2) mice. Groups 3-7 received an allogeneic cell transfer from donor LP/J mice consisting of bone marrow and splenic cells obtained from donor female LP/J (CD45.2) mice. The spleen cells were isolated using the Miltenyi GentleMACS Dissociators. The BM cells were isolated using standard flushing practices. Post-transplant, all animals were housed under standard environmental conditions, and were maintained on the appropriate irradiated sterile rodent chow and sterile water ad libitum. Additionally, following TBI on Day −1 and up to Day 8, all animals were given supplemental high calorie highly palatable food in a dish on the bottom of the cage. Following transfer on Day 0, and then again as needed, all animals were given 1 mL/animal/day of supplemental fluids (warmed ringers solution) via subcutaneous injection. An additional 1 mL fluid may have been given in the pm on an as needed basis.

All animals were monitored on a daily basis to record weight change, survival, and GVHD score (Table 4 of Example 3) and incidence of diarrhea and bloody stool. In addition, a second scoring system for sclerodermatous chronic GVHD was used (Table 5 of Example 3).

On Day 20, animals in Groups 3-7 were redistributed into groups so that average scGVHD score was similar across Groups 3-7. Redistribution was based on the composite, 5-component scGVHD score described in Table 6. Animals were dosed with vehicle or test articles as detailed in Table 6. Dosing started on Day 21 and continued until Day 55. Animals receiving ibrutinib were only administered ibrutinib for the AM dose, followed by vehicle for the PM dose.

On Days 33, 36, 39, 42 and 45, all animals were photographed under isoflurane anesthesia (topdown picture, plain white background) to assess disease severity. Each photo was tagged with the corresponding animal's number to aid identification.

Animals that appeared to be in pain were administered buprenorphine BID, as needed. Animals that were unable to right themselves, cold to the touch or moribund were euthanized. Animals that required euthanasia were euthanized by CO₂ inhalation and did not have organs collected. Animals that were found dead did not have collections performed. On Day 56, all surviving animals were sacrificed by CO₂ inhalation and organs detailed in Table 6 were collected.

TABLE 6
Study Design.

| | | | | | | | Sacrifice & |
|---|---|---|---|---|---|---|---|
| | No. | TBI | Cell Transfer | | | | Collections |
| Group | Animals | Day −1 | Day 0 | Treatment | Schedule | Photo | Day 56 |
| 1 | 6 | — | — | — | — | Days | 8. Blood |
| | | | | | | 33, 36, | (plasma) |
| 2 | 10 | | 1 × 10⁷ bone | Vehicle | BID PO | 39, 42, 45 | 9. Lungs |
| | | | marrow | | D21-55 | | Left (fixed) |
| | | | | | | | Right |
| 3 | 12 | 8.5 Gy | 4 × 10⁶ | Vehicle | | | (frozen) |
| | | | splenocytes | | | | 10. Skin |
| 4 | 10 | | 1 × 10⁷ bone | Ibrutinib | *BID PO | | 1 cm² |
| | | | marrow | 12.5 mg/kg | D21-55 | | (fixed) |
| 5 | 10 | | | Ibrutinib | | | 1 cm² |
| | | | | 25 mg/kg | | | (frozen) |
| 6 | 10 | | | Rilzabrutinib | BID PO | | 4. Spleen |
| | | | | 20 mg/kg | D21-55 | | (frozen) |
| 7 | 10 | | | Rilzabrutinib | | | |
| | | | | 40 mg/kg | | | |

Total number = 68 animals;
*Ibrutinib administered for AM dose followed by 0.5% MC vehicle for PM dose.

### Disease Induction.

All recipient animals received a total body irradiation dose of 8.5 Gy on Day −1 as detailed in Table 6. On Day 0, Group 3-7 animals received a bone marrow (BM) transplant containing bone marrow cells in combination with splenocytes obtained from donor LP/J (CD45.2) mice via the tail vein (200 μL) according to Table 6. On Day 0, Group 2 animals received a bone marrow (BM) transplant containing only bone marrow cells obtained from donor LP/J (CD45.2) mice via the tail vein (200 μL) according to Table 6. The spleen cells were isolated using the Miltenyi Gentlemacs Dissociators. The BM cells were isolated using standard flushing practices (femur and tibia) and counted. Post-transplant, all recipient animals were housed under standard environmental conditions and were maintained on the appropriate rodent chow and sterile water ad libitum.

### Dosing.

Vehicle. The vehicle was 0.5% methylcellulose (MC) administered at a dose of 0.1 mL/20 g. Animals were treated twice daily on Days 21-55.

Ibrutinib. Ibrutinib was formulated with vehicle (0.5% MC). Ibrutinib was administered orally to mice of Group 4 at a dose of 12.5 mg/kg, and orally to mice of Group 5 at a dose of 25 mg/kg. Animals were treated twice daily on Days 21-55 as follows: the AM dose was ibrutinib, and the PM dose was vehicle only.

Rilzabrutinib. Rilzabrutinib was formulated with vehicle (0.5% MC). Rilzabrutinib was administered orally to mice of Group 6 at a dose of 20 mg/kg, and orally to mice of Group 7 at a dose of 40 mg/kg. Animals were treated twice daily on Days 21-55.

Animals in Group 1 were not administered any treatments throughout the study. Animals in Groups 2-7 were administered vehicle or test article by oral gavage (PO) with dosing beginning on Day 21 and administered twice per day (BID), until Day 55. Animals in Groups 4 and 5 were administered ibrutinib for the first/AM dose and vehicle for the last/PM dose each day of dosing. Animals in Groups 2, 3, 6, and 7 received vehicle or test article as indicated in Table 6 for both BID doses.

### In-Life Monitoring and GVHD Assessment.

Animals were observed daily in order to assess possible differences among treatment groups and/or possible toxicity resulting from the treatments. Daily readouts were weight change, survival, standard GVHD score, modified GVHD score, incidence of diarrhea, and incidence of bloody stool.

A clinical score for GVHD was obtained daily for the entire study duration as assessed by a standard scoring system (Table 4) and a modified scoring system for scGVHD (Table 5). The standard GVHD score was based on 5 criteria: percentage of weight change, posture (hunching), activity, fur texture, and skin integrity (maximum index=10). The modified GVHD score for scGVHD was based on 5 criteria: percentage of weight change (0-4), posture (hunching) (0-3), activity (0-3), fur texture (0-4), and skin integrity (0-4) (maximum index=18). The overall score was reported, as well as the scores for each individual parameter. Animals that appeared to be in pain were administered buprenorphine BID, as needed.

On Days 33, 36, 39, 42 and 45, all animals were photographed under isoflurane anesthesia (topdown picture, plain white background) to assess disease severity. Each photo was tagged with the corresponding animal's number to aid identification.

### Supportive Care and Euthanasia Criteria

Following TBI on Day −1 up to Day 8, all animals were given supplemental high-calorie highly palatable food in a dish on the bottom of the cage. Following transfer on Day 0, and then again as needed, all animals were given 1 mL/animal/day of supplemental fluids (warmed ringers solution) via subcutaneous injection. An additional 1 mL fluid may have been given in the afternoon on an as-needed basis. Any animal that exceeded 15% body weight loss were given highly palatable soft food and 1 mL supplemental fluids (warmed ringers solution) via subcutaneous injection once per day. Any animal that exceeded 20% body weight loss were given 1 mL supplemental fluids (warmed ringers solution) via subcutaneous injection twice per day. Animals that appeared to have been in pain were administered buprenorphine BID, as needed. Any animal that lost >30% of its body weight, showed an inability to eat, were unable to right themselves, were cold to the touch, or was moribund was euthanized. Animals that were found dead or were euthanized prior to scheduled termination days did not undergo terminal collections.

### Sacrifice and Sample Collection.

At the conclusion of the study (Day 56), all surviving animals were sacrificed by xylazine overdose and organs detailed in Table 6 were collected. Blood, lungs, skin, and spleen were collected at sacrifice.

Blood. Prior to sacrifice, ~0.2 mL of blood was collected from all animals by retro-orbital bleed into K₂EDTA tubes. The blood was centrifuged, and the plasma collected and stored at −80° C.

Lung. The lungs were removed and whole lung and right lung were weighed. The right lung was tied off on the right bronchus, excised below the tie, snap frozen in liquid nitrogen and stored at −80° C. The left lung was insufflated with 10% Neutral Buffered Formalin (NBF), tied off on the trachea to maintain insufflation, fixed in 10% NBF for 24 hours, then moved to PBS prior to processing for histopathology.

Skin. Prior to collection, skin was shaved. A 1×1 cm portion of the skin (from the back of the animal, between the shoulders, closer to head) was excised, trimmed of any excess fat and/or connective tissue, sandwiched between foam in a tissue cassette, and placed in formalin. After 24 hours, the skin was transferred to PBS for storage until subsequent histological analyses. A second 1 cm×1 cm portion of the skin (from the back of the animal, between the shoulders, just below the first piece) was excised, trimmed of any excess fat and/or connective tissue, weighed, flash frozen, and stored at −80° C. If a visible lesion was present, it was collected in a way that captured part of it in both fixed and frozen sections.

Spleen. The spleen was excised and trimmed of any excess connective tissue. The spleen was then weighed, flash frozen, and stored at −80° C.

### Survival and Progression-Free Survival.

Animal deaths were evaluated during the course of the study. In this model, animal deaths are commonly attributable to severe disease. In this study, eight (8) animals were either found dead or were euthanized due to either euthanasia criteria or moribundity. Progression-free survival was defined as an increase in GVHD Score of less than or equal to 2 in comparison to Day 21 GVHD Score (standard or modified). A difference in GVHD score of 3 or higher relative to Day 21 meant that the animal no longer is in progression-free survival and had begun disease progression.

### Skin, and Lung Histopathology.

Fixed lung and skin samples were embedded in paraffin, sectioned at 5 microns, and slides stained with hematoxylin and eosin (H&E). One slide per block was sectioned and stained with hematoxylin and eosin (H&E). All slides were evaluated and scored with light microscopy by a board-certified veterinary pathologist.

### Outcome Evaluation.

Study endpoints were body weight change, survival, progression-free survival, standard GVHD score, modified scGVHD score, and histopathology.

### Statistical Analyses.

Parametric data (weight change) were evaluated using one-way ANOVA with Dunnett's multiple comparisons test to compare all groups to the vehicle control group. Non-parametric data (GVHD scores) were analyzed with Kruskal-Wallis with Dunn's post-hoc test. All statistical analyses were performed using GraphPad Prism 8.4.3 Software (La Jolla, CA). Statistical significance was achieved when p<0.05.

### Results.

Survival. Survival was tracked for all animals during the course of the study and the percent survival is shown in FIG. 4.

All naïve animals (Group 1) survived to the conclusion of the study. Two animals died prior to the start of treatment: one animal that received BM only (Group 2) and one animal that received BM with splenocytes. After the start of treatment on Day 21, all vehicle-treated BM only animals (Group 2) and 12.5 mg/kg ibrutinib treated diseased animals (Group 4) survived until the end of the study. In diseased animals, survival was 90% with 25 mg/kg ibrutinib treatment (Group 5), 90% with 40 mg/kg rilzabrutinib treatment (Group 7), 82% in vehicle-treated animals (Group 3), and 80% in 20 mg/kg rilzabrutinib treated animals (Group 6).

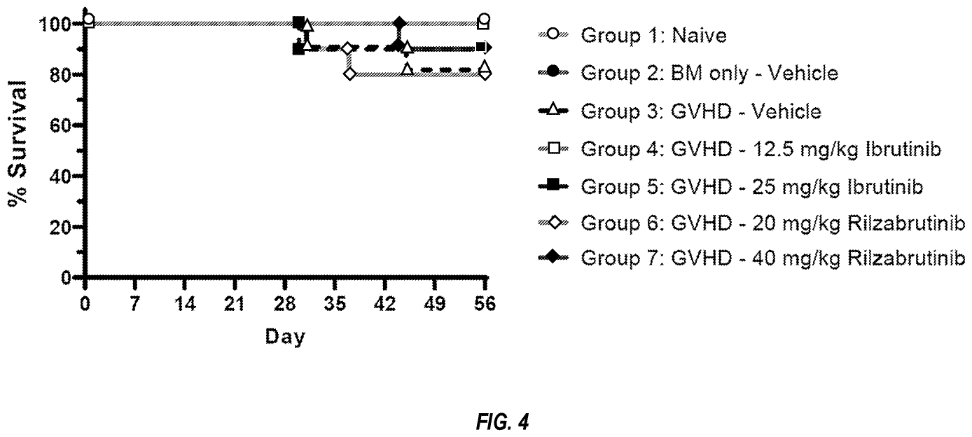

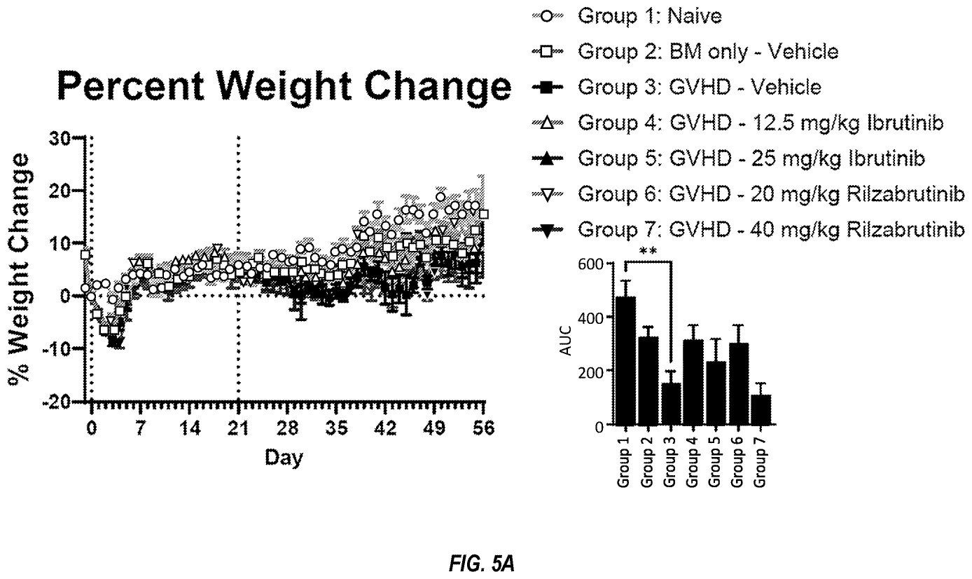

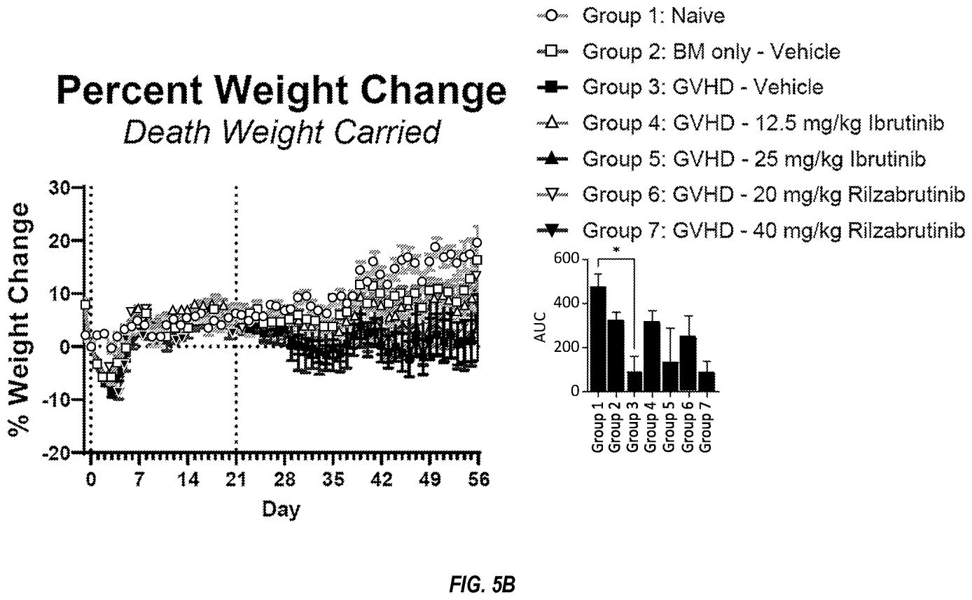

Body Weight. GVHD disease induction (cell transfer of splenocytes and bone marrow cells) prevented normal weight gain in all diseased animals. Survival past Day 14 indicates successful engraftment of donor cells. Percent weight change is plotted in FIG. 5A and percent weight change with death weight carried forward is plotted in FIG. 5B. Naïve animals (Group 1) displayed normal increases in weight throughout the study. By AUC analysis, naïve animals (Group 1) displayed significantly more weight gain (p<0.01) compared to vehicle-treated animals (Group 2). Although not statistically significant, BM only animals treated with vehicle (Group 2) displayed more weight gain than diseased animals treated with vehicle (Group 3). There were no significant differences in weight change observed between diseased animals treated with test articles (Groups 4-7) and vehicle-treatment (Group 3). However, diseased animals treated with 12.5 mg/kg ibrutinib (Group 4) and 20 mg/kg rilzabrutinib (Group 6) had notably more weight gain than vehicle treatment (Group 3).

In order to account for changes in average weight due to deaths in the study which contribute to survivor bias, death weight was carried out until Day 56 and is presented in FIG. 5B. By AUC analysis from Day 0, naïve animals (Group 1) displayed significantly more weight gain (p<0.05) compared to BM only vehicle-treated animals (Group 2). After adjusting for survivor bias, BM only vehicle-treated animals (Group 2) continued to display notably more weight gain compared to diseased animals treated with vehicle (Group 3). By AUC analysis, no significant differences in weight change were observed between diseased animals treated with test articles (Groups 4-7) and vehicle-treatment (Group 3). The trend of increased weight gain in diseased animals treated with 12.5 mg/kg ibrutinib (Group 4) and 20 mg/kg rilzabrutinib (Group 6) compared to vehicle treatment (Group 3) remained after adjusting for survivor bias.

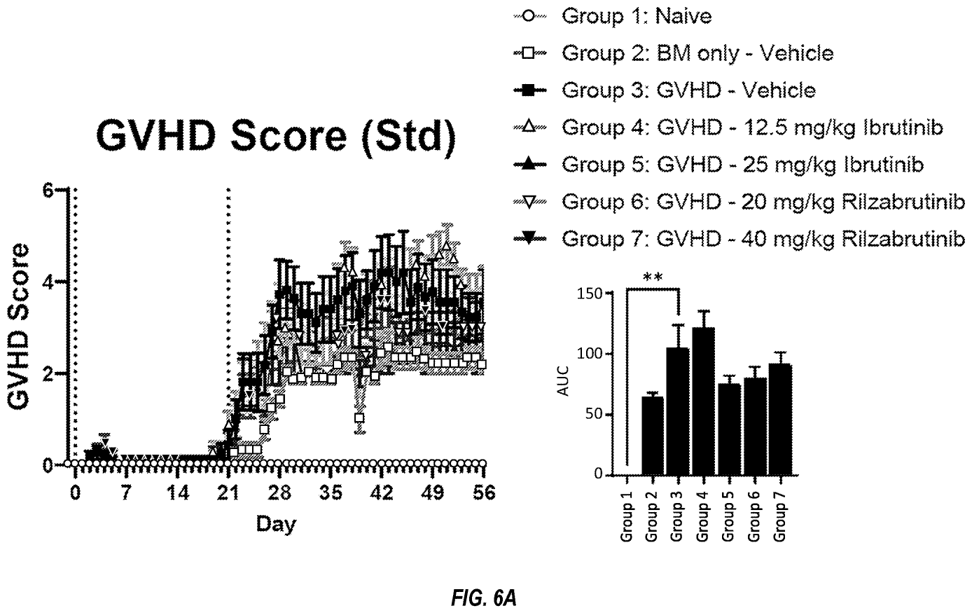

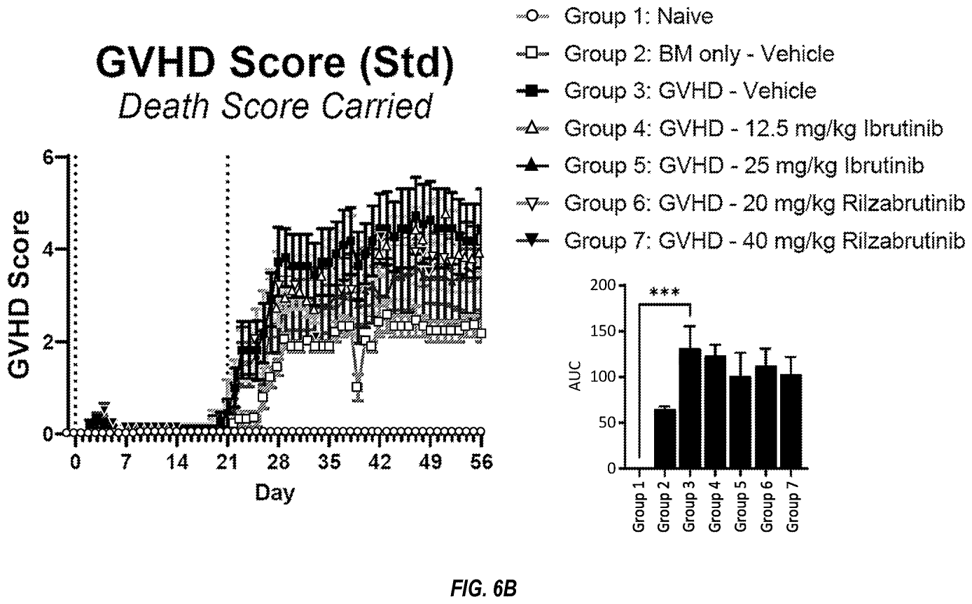

GVHD Score—Standard Scale. GVHD disease induction (cell transfer of splenocytes and bone marrow cells) induced disease in all animals in Groups 3-7 as assessed using the multi-parameter GVHD scoring systems shown in Table 4. Survival of animals past Day 14 verifies successful engraftment of the transplanted cells. Standard GVHD scores are displayed in FIG. 6A. By AUC analysis from Day 0, naïve animals (Group 1) displayed significantly lower standard GVHD scores (p<0.01) compared to vehicle-treated diseased animals (Group 3). Although not statistically significant, BM only animals treated with vehicle (Group 2) displayed lower standard GVHD scores than diseased animals treated with vehicle (Group 3). There were no significant differences in standard GVHD scores observed between diseased animals treated with test articles (Groups 4-7) and vehicle-treatment (Group 3). However, diseased animals treated with 25 mg/kg ibrutinib (Group 5), 20 mg/kg rilzabrutinib (Group 6), and 40 mg/kg rilzabrutinib (Group 7) had notably lower standard GVHD scores than vehicle treatment (Group 3).

In order to account for changes in GVHD score due to deaths in the study which contribute to survivor bias, death score was carried out until Day 56 and is presented in FIG. 6B. By AUC analysis from Day 0, naïve animals (Group 1) displayed significantly lower standard GVHD scores (p<0.001) with death scores carried forward when compared to vehicle-treated diseased animals (Group 3). After adjusting for survivor bias, BM only animals treated with vehicle (Group 2) continued to display notably lower standard GVHD scores than diseased animals treated with vehicle (Group 3). There were no significant differences, in standard GVHD score with death scores carried forward, observed between diseased animals treated with test articles (Groups 4-7) and vehicle-treatment (Group 3). The trend of lowered standard GVHD scores in diseased animals treated with 25 mg/kg ibrutinib (Group 5), 20 mg/kg rilzabrutinib (Group 6), and 40 mg/kg rilzabrutinib (Group 7) compared to vehicle treatment (Group 3) was dampened after adjusting for survivor bias.

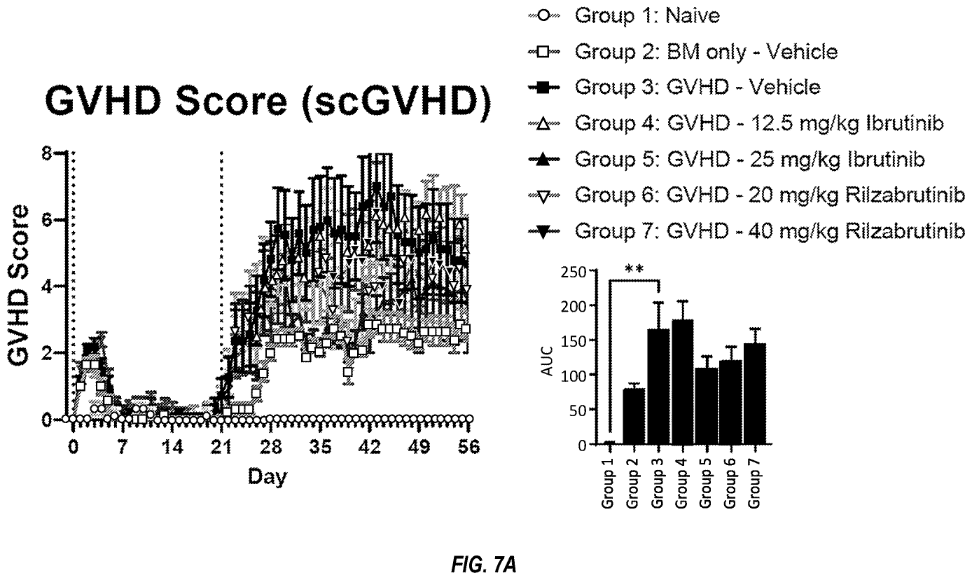

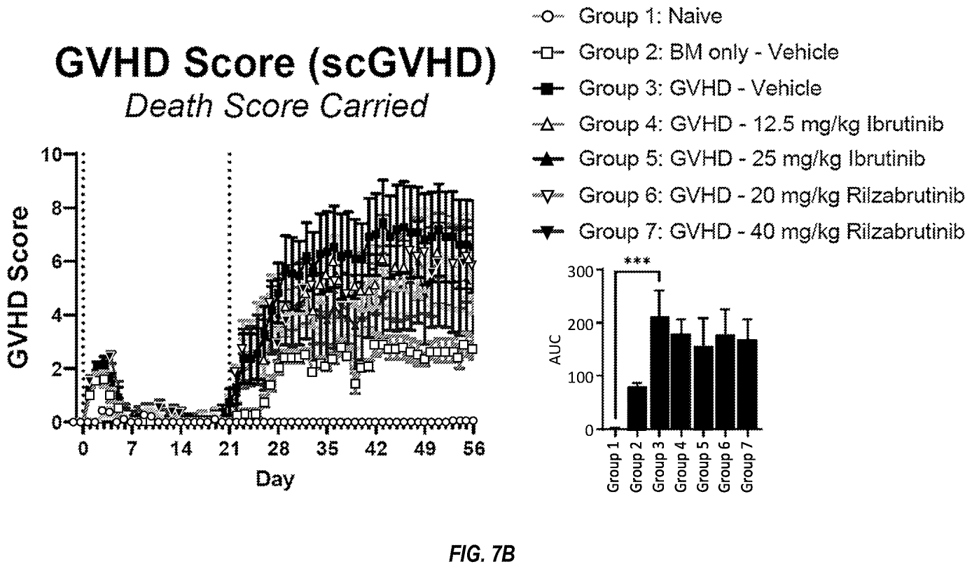

GVHD Score—Modified Scale. GVHD disease induction (cell transfer of splenocytes and bone marrow cells) induced disease in all animals in Groups 3-7 as assessed using a modified multi-parameter GVHD scoring systems shown in Table 5, for sclerodermatous GVHD. Survival of animals past Day 14 verifies successful engraftment of the transplanted cells. Modified GVHD scores are displayed in FIG. 7A. By AUC analysis from Day 0, naïve animals (Group 1) displayed significantly lower modified scGVHD scores (p<0.01) compared to vehicle-treated diseased animals (Group 3). Although not statistically significant, BM only animals treated with vehicle (Group 2) displayed lower modified GVHD scores compared to diseased animals treated with vehicle (Group 3). There were no significant differences in modified GVHD scores observed between diseased animals treated with test articles (Groups 4-7) and vehicle-treatment (Group 3). However, diseased animals treated with 25 mg/kg ibrutinib (Group 5), 20 mg/kg rilzabrutinib (Group 6), and 40 mg/kg rilzabrutinib (Group 7) had notably lower modified GVHD scores than vehicle treatment (Group 3).

In order to account for changes in average scGVHD score due to deaths in the study which contribute to survivor bias, death score was carried out until Day 56 and is presented in FIG. 7B. By AUC analysis, when death score was carried forward, naïve animals (Group 1) displayed significantly lower modified GVHD scores (p<0.001) compared to vehicle-treated diseased animals (Group 3). After adjusting for survivor bias, BM only animals treated with vehicle (Group 2) continued to display notably lower modified GVHD scores than diseased animals treated with vehicle (Group 3). There were no significant differences, in modified GVHD score with death scores carried forward, observed between diseased animals treated with test articles (Groups 4-7) and vehicle-treatment (Group 3). The trend of lowered modified GVHD scores in diseased animals treated with 25 mg/kg ibrutinib (Group 5), 20 mg/kg rilzabrutinib (Group 6) and 40 mg/kg rilzabrutinib (Group 7) compared to vehicle treatment (Group 3) was dampened after adjusting for survivor bias.

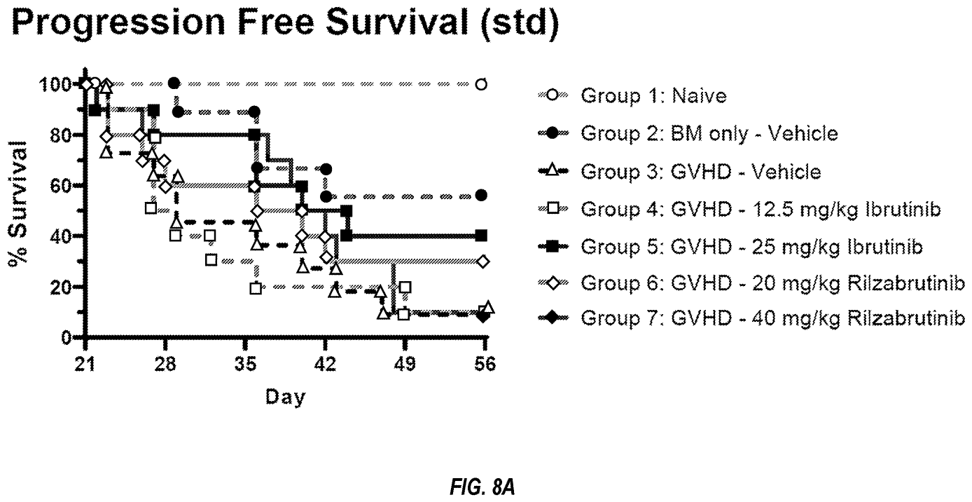

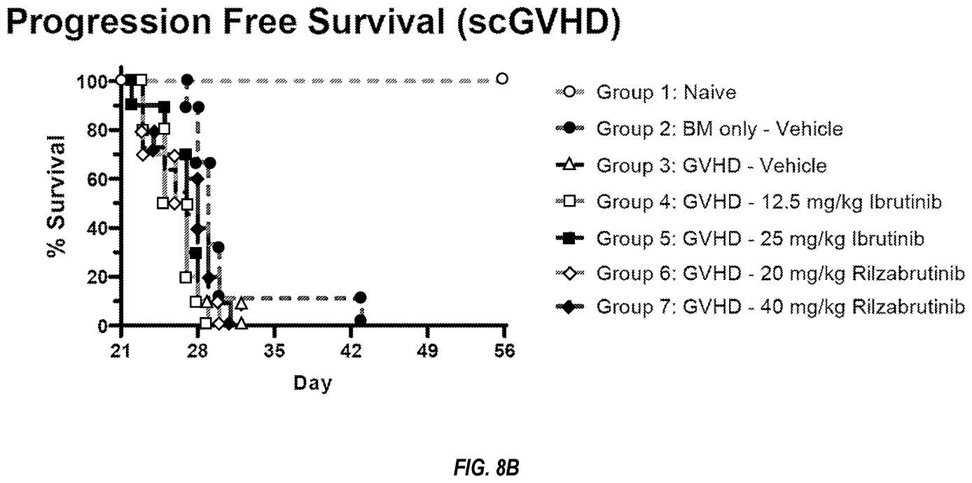

Progression-Free Survival. Progression free survival (PFS) was tracked for all animals during the course of the study and the percent progression free survival was plotted, as shown in FIG. 8A (standard GVHD scale) and FIG. 8B (modified scGVHD scale). Progression free survival was defined as an increase in GVHD Score of less than or equal to 2 in comparison to Day 21 GVHD Score (standard or modified). Disease progression was defined as an increase in GVHD Score of greater than 2 in comparison to Day 21 GVHD Score (standard or modified). Under both metrics, animals in which GVHD was induced displayed reduced PFS as compared to naïve animals (Group 1), all of which did not present with disease progression. Analysis using the standard GVHD scoring scale showed that 56% of the BM only animals treated with vehicle (Group 2) did not display progressive disease by Day 56. In GVHD animals, 40% of 25 mg/kg ibrutinib treated animals (Group 5), 30% of 20 mg/kg rilzabrutinib (Group 6), 10% of 40 mg/kg rilzabrutinib (Group 7), 10% of 12.5 mg/kg ibrutinib (Group 4), and 9% of vehicle treated animals (Group 3) did not display progressive disease by Day 56. Due to the granularity of the modified scoring scale, analysis using the modified scGVHD scale showed animals that received BM only (Group 2) displayed progressive disease by Day 43 and animals that received BM with splenocytes (Groups 4-7) displayed progressive disease by Day 32.

Skin and Lung Histopathology. Fixed skin, and left lung samples from surviving animals (n=60) were embedded in paraffin and sectioned at 5 microns. One slide per block was sectioned and stained with H&E. All slides were evaluated and scored with light microscopy by a board-certified veterinary pathologist. Lesions in both the lung and skin were assigned a severity score based on the extent of findings on an ordinal range of 0-5, where 0=not present/within normal limits, 1=minimal, 2=mild, 3=moderate, 4=marked, 5=severe.
Grade 0: Within normal limits
Grade 1: Minimal; <10% of tissue compartment affected or minimal diffuse change
Grade 2: Mild; 10-25% of tissue compartment affected or mild diffuse change
Grade 3: Moderate; 26-50% of tissue compartment affected
Grade 4: Marked; 51-75% of tissue compartment affected
Grade 5: Severe; >75% of tissue compartment affected
   In each organ, histologic feature scores were added together to obtain a sum score. Lung range: 0-10; skin range: 0-30.

Dermal Thickness Measurements. Using H&E-stained slides, the dermis was measured (μm) in five areas across the tissue, with a mean calculated for each sample. Measurements were performed from epidermal basement membrane to the most superficial border of dermal collagen with subcuticular adipose. These measurements were only performed in areas of non-tangentially sectioned tissue, free of histologic artifacts, and with an intact epidermis.

Statistical Analysis. Data are presented as mean±standard error of the mean (SEM). Semi-quantitative sum and component histopathology scores were analyzed (GraphPad Prism) by non-parametric ANOVA (Kruskal-Wallis H test) with Dunn's multiple comparison post-hoc tests; dermal measurements were analyzed by one-way ANOVA with post-hoc Tukey tests as appropriate. Significance was set at p<0.05 for all tests.

### Results and Discussion.

### Morphologic Pathology (H&E)

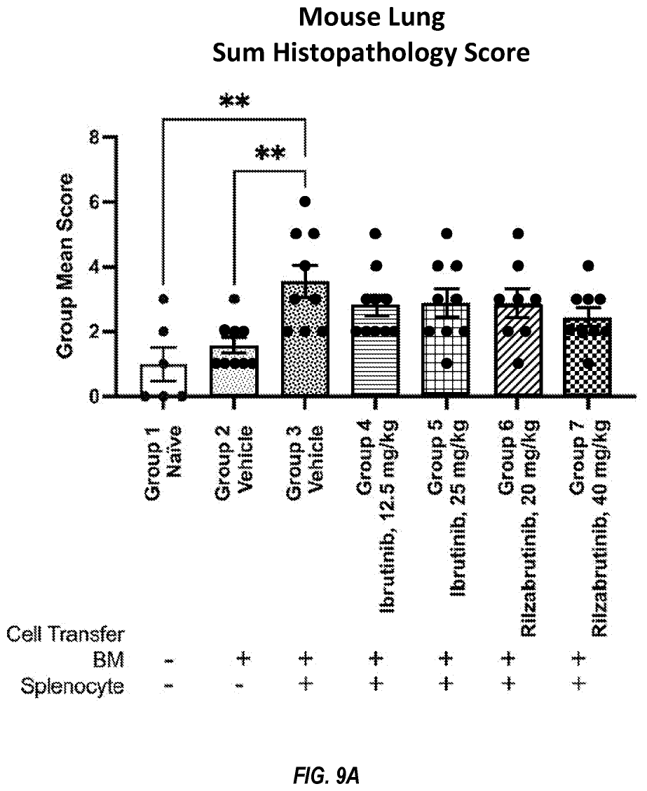

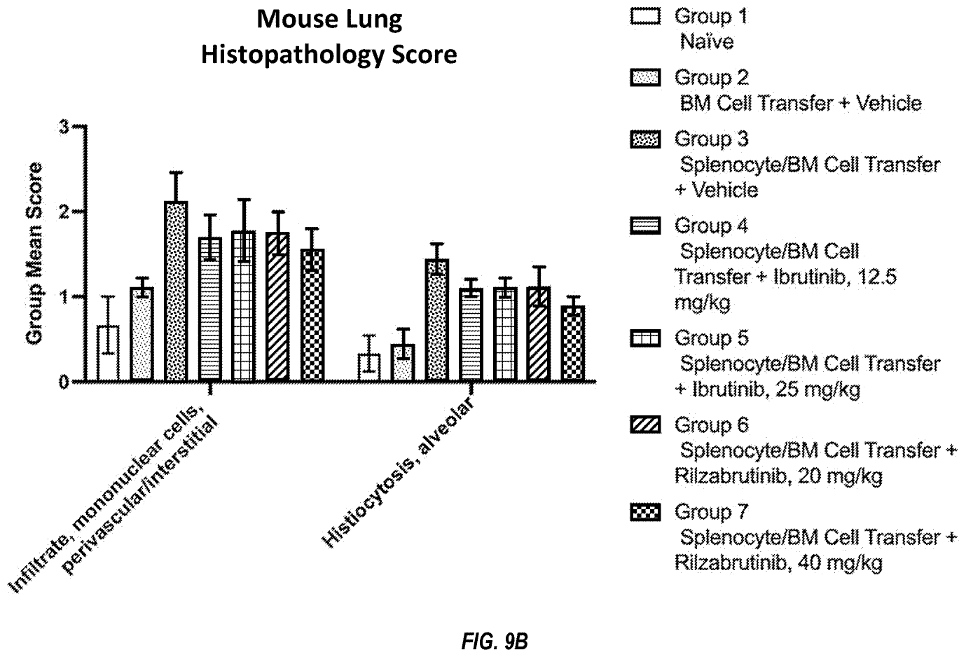

Lung. Histologic findings in pulmonary tissues were consistent for those expected in the chronic GVHD model. The primary finding was infiltration of mononuclear cells (lymphocytes dominant, with fewer plasma cells and macrophages; rare neutrophils seen) in perivascular and less frequently, peribronchiolar zones. Mononuclear cell infiltrates were typically seen surrounding the largest caliber blood vessels, with more severely affected samples exhibiting dense cuffing (up to 15 cells thick) from large to small caliber blood vessels. Extension into the adjacent interstitium was infrequently observed. Increased numbers of alveolar macrophages were also consistently seen, sometimes in areas with interstitial infiltration, but more often along the alveoli of the periphery. Sporadically, naïve mice (Group 1) had minimal to mild perivascular/bronchiolar infiltrates of mononuclear cells (consistent with bronchiolar-associated lymphoid tissue), with similar findings in mice given bone marrow cell transfers only (Group 2). Significantly increased sum histopathology scores (FIG. 9A) were observed in animals given bone marrow and splenocyte cell transfers (Group 3) compared to bone marrow only (Group 2; p=0.01) or naïve animals (Group 1; p=0.007). Both perivascular mononuclear cells and alveolar histiocytosis scores (FIG. 9B) were increased in GVHD mice. Small reductions in lung sum (FIG. 9A) and individual component histopathology scores (FIG. 9B) were observed with either ibrunitib (Groups 4 and 5) or rilzabrutinib (Groups 6 and 7) compared to the vehicle, but no comparisons reached statistical significance.

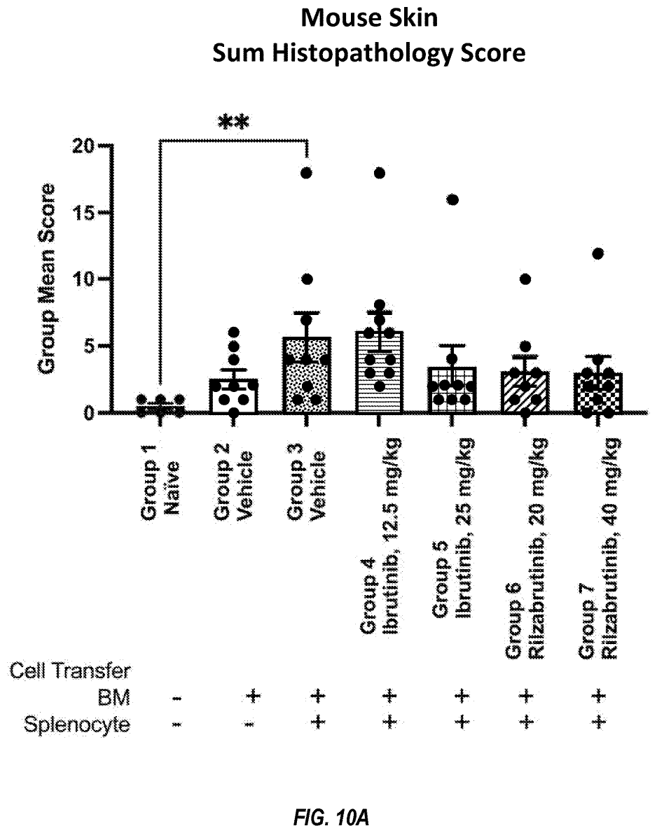

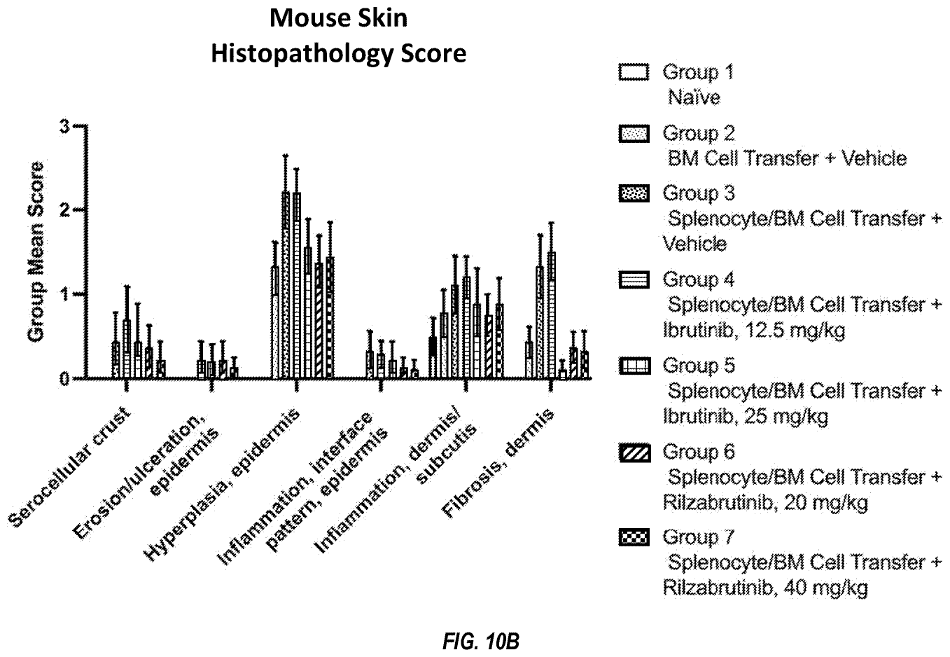

Skin. Histologic findings in the skin varied considerably in severity across animals receiving cell transfers and within groups. The range of histologic findings included serocellular crusting, epidermal erosion or ulceration, epidermal and follicular hyperplasia, mononuclear inflammatory cell infiltration both within the epidermis/follicle (interface dermatitis pattern) and within the dermis, and dermal fibrosis. Note that the primary findings, seen most consistently, included minimal to mild epidermal hyperplasia, dermal mononuclear cell infiltration, and dermal fibrosis; crusting, significant necrosis with ulceration, interface pattern epidermal inflammation, and significant epidermal and follicular hyperplasia were sporadically observed. Serocellular crusts were characterized by the accumulation of proteinaceous fluid, necrotic debris, and degenerative neutrophils on the epidermal surface, with or without epidermal necrosis, which was typically seen in short segments either partial thickness (erosion) or full thickness, sometimes extending into the subcutis (ulceration). Epidermal hyperplasia was characterized by thickening of the epidermis by increased nucleated keratinocyte layers, 3-8 cells thick. Mononuclear cell (lymphocyte) infiltration in the superficial dermis rarely extended into the epidermis or hair follicles, accompanied by necrotic/apoptotic keratinocytes in the interface-pattern lesion, with rupture of hair follicles infrequently observed; this latter finding was accompanied by infiltrates of neutrophils and macrophages surrounding keratin or hair shafts. In most samples, mononuclear cell infiltration was largely seen in the dermis, with inconsistent association with regions of hyperplastic epidermis and areas of dermal fibrosis, which was characterized by the deposition of collagen bundles or amphophilic extracellular matrix in parallel, distorting normal dermal collagen organization. Dermal fibrosis and inflammation caused slight thickening of the dermis. Histologic findings were minimal in naïve mice (Group 1), with more severe findings seen with the addition of splenocyte cell transfers (Group 3) compared to bone marrow alone (Group 2); however, sum scores (FIG. 10A) were only significantly induced by bone marrow+splenocyte transfer (Group 3) compared to naïve (Group 1; p=0.006). Administration of ibrunitib at 25 mg/kg (Group 5) or either dose of rilzabrunitib (Groups 6 and 7) was associated with lowered sum scores (FIG. 10A) than treatment with the vehicle (Group 3); these changes were driven by several features, but epidermal hyperplasia and dermal fibrosis score (FIG. 10B) differences were the best developed, including statistically lowered dermal fibrosis scores for ibrunitib (25 mg/kg; Group 5; p=0.02) and a statistical trend toward lowered scores seen with rilzabrutinib at 40 mg/kg (Group 7; p=0.09) compared to vehicle (Group 3).

### Dermal Thickness Measurements (H&E)

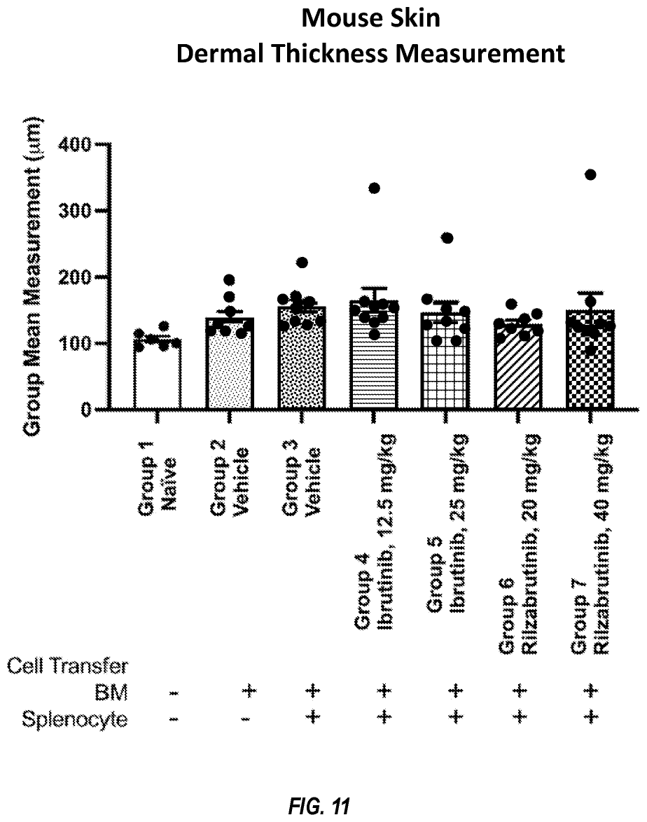

Skin from näive mice (Group 1) was within normal limits, with minimal within-group variability in dermal thickness measurements (FIG. 11). Cell transfers of bone marrow (Group 2) or bone marrow with splenocytes (Group 3) were associated slightly greater dermal thickness, associated with inflammatory cell infiltration and fibrosis. Test article treatment minimally affected dermal thickness with a small reduction in mean thickness seen in animals given rilzabrutinib at 20 mg/kg (Group 6). Dermal thickness measurements did not achieve among-group significance (p=0.3) in this study.

### Conclusions.

All naïve animals survived to the conclusion of the study. After the start of treatment on Day 21, all vehicle-treated BM only animals and 12.5 mg/kg ibrutinib treated diseased animals survived until the end of the study. In diseased animals, survival was 90% with 25 mg/kg ibrutinib treatment, 90% with 40 mg/kg rilzabrutinib treatment, 82% in vehicle treated animals, and 80% in 20 mg/kg rilzabrutinib treated animals.

By AUC analysis, naïve animals displayed significantly more weight gain compared to vehicle-treated diseased animals. After adjusting for survivor bias, BM only vehicle treated animals continued to display notably more weight gain compared to diseased animals treated with vehicle. There were no significant differences in weight change observed between diseased animals treated with test articles and vehicle-treatment. The trend of increased weight gain in diseased animals treated with 12.5 mg/kg ibrutinib and 20 mg/kg rilzabrutinib compared to vehicle treatment remained after adjusting for survivor bias.

By AUC analysis, naïve animals displayed significantly lower standard GVHD scores with death scores carried forward when compared to vehicle-treated diseased animals. After adjusting for survivor bias, BM only animals treated with vehicle continued to display notably lower standard GVHD scores than diseased animals treated with vehicle. There were no significant differences, in standard GVHD score with death scores carried forward, observed between diseased animals treated with test articles and vehicle treatment. The trend of lowered standard GVHD scores in diseased animals treated with 25 mg/kg ibrutinib, 20 mg/kg rilzabrutinib, and 40 mg/kg rilzabrutinib compared to vehicle treatment was dampened after adjusting for survivor bias.

By AUC analysis, when death score was carried forward, naïve animals displayed significantly lower modified GVHD scores compared to vehicle-treated diseased animals. After adjusting for survivor bias, BM only animals treated with vehicle continued to display notably lower modified GVHD scores than diseased animals treated with vehicle. There were no significant differences, in modified GVHD score with death scores carried forward, observed between diseased animals treated with test articles and vehicle treatment. The trend of lowered modified GVHD scores in diseased animals treated with 25 mg/kg ibrutinib, 20 mg/kg rilzabrutinib, and 40 mg/kg rilzabrutinib compared to vehicle treatment was dampened after adjusting for survivor bias.

Näive animals displayed no disease progression based on the standard and modified GVHD scoring scales for PFS. Analysis using the standard GVHD scoring scale showed that 56% of the BM only animals treated with vehicle did not display progressive disease by Day 56. While in GVHD animals, 40% of 25 mg/kg ibrutinib treated animals, 30% of 20 mg/kg rilzabrutinib, 10% of 40 mg/kg rilzabrutinib, 10% of 12.5 mg/kg ibrutinib, and 9% of vehicle-treated animals did not display progressive disease by Day 56. Due to the granularity of the modified scoring scale, analysis using the modified scGVHD scale showed animals that received BM only displayed progressive disease by Day 43 and all animals that received BM with splenocytes displayed progressive disease by Day 32.

Perivascular mononuclear cell infiltrates were the primary lung finding, with an array of findings in the skin, including epidermal and follicular hyperplasia, mononuclear cell infiltrates, and dermal fibrosis, along with the sporadic development of more severe findings including epidermal necrosis/ulceration, interface dermatitis-pattern mononuclear cell infiltration in the diseased animals. Treatment with either ibrunitib or rilzabrutinib produced small changes in the severity of lung and skin lesion severity scores compared to vehicle treatment; however, dermal fibrosis score reductions reached statistical significance in diseased animals treated with 25 mg/kg ibrutinib or trended toward statistical significance in diseased animals treated with 40 mg/kg rilzabrutinib.

Administration of splenocyte+bone marrow cell transfers from LP/J donor mice to irradiated C57Bl/6 mice in a model of chronic GVHD was associated with the development of histologic lesions in the lung and skin. Perivascular mononuclear cell infiltrates were the primary lung finding, with an array of findings in the skin, including epidermal and follicular hyperplasia, mononuclear cell infiltrates, and dermal fibrosis, along with the sporadic development of more severe findings including epidermal necrosis/ulceration, interface dermatitis-pattern mononuclear cell infiltration.

Treatment with either ibrunitib or rilzabrutinib produced small changes in the severity of lung and skin lesion severity scores compared to vehicle treatment; however, dermal fibrosis score reductions reached (ibrunitib) or trended toward (rilzabrutinib) statistical significance in this study.

### Example 5. Activity of BTK Inhibitors Belumosudil, Rilzabrutinib, and Ibrutinib in a Mouse Model of Sclerodermatous Chronic Graft-Versus-Host Disease (cGVHD)

The main objective of this study was to evaluate the effect of combinations of select BTK inhibitors on a murine model of sclerodermatous chronic graft-versus-host disease (GVHD). However, as shown herein, the data is inconclusive because of multiple study limitations. Among said limitations, the disease induction was not robust and the positive controls failed to show a significant difference. Thus, treatments expected to show disease modifying activity in the model did not do so.

### Animals.

Recipient female C57Bl/6 (CD45.1) mice (n=78; 8 weeks) with an average starting body weight (±SEM) of 18.55±0.13 g were obtained from Jackson Laboratories (Bar Harbor, Maine). Donor female LP/J mice (n=108; 6-10 weeks) were obtained from Jackson Laboratories (Bar Harbor, Maine). Animals were acclimatized prior to study commencement. During this period, the animals were observed daily in order to reject any that presented in poor condition.

The study was performed in animal rooms provided with HEPA filtered air at a temperature of 70±5° F. and 50%±20% relative humidity. Animals were housed in groups of 6-12 per cage. Animal rooms were set to maintain a minimum of 12 to 15 air changes per hour. The room was on an automatic timer for a light/dark cycle of 12 hours on and 12 hours off with no twilight. Alpha-dri® or equivalent bedding was used. Cages, tops, and water bottles were washed with a commercial detergent and allowed to air dry. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number, and treatment group was used to mark all cages. The temperature and relative humidity were recorded during the study and the records retained.

Animals were fed with LabDiet 5053 rodent diet and water was provided ad libitum. Animals were randomized into eight (8) groups at the start of the study: one (1) group of six (6) animals, one (1) group of 12 animals, and six (6) groups of ten (10) animals each. Each animal was identified by an ear punch corresponding to an individual number. A cage card was used to identify each cage and was marked with the study number, treatment group number, and animal numbers.

### Study Design.

Sclerodermatous chronic GVHD was induced in C57Bl/6 (CD45.1) mice using a single acute dose of 8.5 Gy of total body irradiation (TBI) on Day −1. On Day 0, the C57Bl/6 (CD45.1) recipients were given an intravenous (IV) injection of a combination of splenocytes and bone marrow cells in sterile 1×PBS: Group 1 served as the Naïve control group and did not receive either TBI or a cell transfer. Group 2 received an allogeneic cell transfer from donor LP/J mice consisting of only bone marrow cells obtained from donor female LP/J (CD45.2) mice. Groups 3-8 received an allogeneic cell transfer from donor LP/J mice consisting of bone marrow and splenic cells obtained from donor female LP/J (CD45.2) mice. The spleen cells were isolated using the Miltenyi GentleMACS Dissociators. The BM cells were isolated using standard flushing practices. Post-transplant, all animals were housed under standard environmental conditions, and were maintained on the appropriate irradiated sterile rodent chow and sterile water ad libitum.

Additionally, following TBI on Day −1 and up to Day 8, all animals were given supplemental high calorie highly palatable food in a dish on the bottom of the cage. Following transfer on Day 0, and then again as needed, all animals were given 1 mL/animal/day of supplemental fluids (warmed ringers solution) via subcutaneous injection. An additional 1 mL fluid was given in the pm on an as needed basis.

All animals were monitored on a daily basis to record weight change, survival, GVHD score (Table 4), and incidence of diarrhea and bloody stool. In addition, a second scoring system for sclerodermatous chronic GVHD (scGVHD) was used (Table 5).

On Day 20, animals in Groups 3-8 were redistributed into groups so that average scGVHD score was similar across Groups 3-8. Redistribution was based on the composite, 5-component scGVHD score described in Table 5.

Animals were dosed with vehicle or test articles as detailed in Table 7, below. Dosing started on Day 21 and continued until Day 55. Animals that received ibrutinib were only administered ibrutinib for the AM dose, followed by vehicle for the PM dose.

On Days 33, 36, 39, 42, and 45 all animals were photographed under isoflurane anesthesia (top-down picture, plain white background) to assess disease severity. Each photo was tagged with the corresponding animal's number to aid identification.

Animals that appeared to be in pain were administered Buprenorphine BID, as needed. Animals that were unable to right themselves, cold to the touch or moribund were euthanized. Animals requiring euthanasia were euthanized by CO₂ inhalation and did not have collections performed. Animals that were found dead did not have collections performed.

On Day 56, all surviving animals were sacrificed by CO₂ inhalation and organs were collected. The details of the study design are shown in Table 7.

TABLE 7
Study Design.

| | | | | | | | Sacrifice & |
|---|---|---|---|---|---|---|---|
| | No. | TBI | Cell Transfer | | | | Collections |
| Group | Animals | Day −1 | Day 0 | Treatment | Schedule | Photo | Day 56 |
| 1 | 6 | — | — | — | — | Days | 1. Blood |
| | | | | | | 33, 36, | (plasma) |
| 2 | 10 | 8.5 Gy | 1 × 10⁷ bone | Vehicle | *BID PO | 39, 42, 45 | 2. Lungs |
| | | | marrow | | D21-55 | | Left (fixed) |
| 3 | 12 | | 4 × 10⁶ | Vehicle | | | Right |
| | | | splenocytes | | | | (frozen) |
| 4 | 10 | | 1 × 10⁷ bone | Belumosudil | | | 3. Skin |
| | | | marrow | *125 mg/kg | | | 1 cm² |
| 5 | 10 | | | Ibrutinib | | | (fixed) |
| | | | | 20 mg/kg | | | 1 cm² |
| 6 | 10 | | | Rilzabrutinib | | | (frozen) |
| | | | | 40 mg/kg | | | |
| 7 | 10 | | | Belumosudil | | | 4. Spleen |
| | | | | **125 mg/kg + | | | (frozen) |
| | | | | Ibrutinib | | | |
| | | | | 20 mg/kg | | | |
| 8 | 10 | | | Belumosudil | | | |
| | | | | **125 mg/kg + | | | |
| | | | | Rilzabrutinib | | | |
| | | | | 40 mg/kg | | | |

Total number = 78 animals;
*Belumosudil and/or ibrutinib administered for AM dose followed by 0.5% MC vehicle for PM dose;
**Animals were dosed with 125 mg/kg belumosudil from Days 21-32. Due to weight loss observed in the belumosudil treated animals (Group 4), the dose of belumosudil for Groups 4, 7, and 8 was decreased to 100 mg/kg. Animals in Groups 4, 7, and 8 were dosed with 100 mg/kg belumosudil from Days 33-55.

### Disease Induction.

All recipient animals received a total body irradiation dose of 8.5 Gy on Day −1 as detailed in Table 7. On Day 0, Group 3-8 animals received a bone marrow (BM) transplant containing bone marrow cells in combination with splenocytes obtained from donor LP/J (CD45.2) mice via the tail vein (200 μL) according to Table 7. On Day 0, Group 2 animals received a bone marrow (BM) transplant containing only bone marrow cells obtained from donor LP/J (CD45.2) mice via the tail vein (200 L) according to Table 7. The spleen cells were isolated using the Miltenyi Gentlemacs Dissociators. The BM cells were isolated using standard flushing practices (femur and tibia) and counted. Post-transplant, all recipient animals were housed under standard environmental conditions and were maintained on the appropriate rodent chow and sterile water ad libitum.

Vehicle. The vehicle was 0.5% methylcellulose (MC) administered at a dose of 0.1 mL/20 g. Animals were treated twice daily (BID) on Days 21-55.

Belumosudil. Belumosudil was formulated with vehicle (0.5% MC). Belumosudil was administered orally to mice of Groups 4, 7, and 8 at a dose of 125 mg/kg on Days 21-32 and at a dose of 100 mg/kg on Days 33-55. Animals were treated twice daily (BID) on Days 21-55 as follows: for Group 4, the AM dose was belumosudil, and the PM dose was vehicle only; for Group 8, rilzabruitinib was formulated in combination with belumosudil for the AM dose.

Ibrutinib. Ibrutinib was formulated with vehicle (0.5% MC). Ibrutinib was administered orally to mice of Groups 5 and 7 at a dose of 20 mg/kg. Animals were treated twice daily (BID) on Days 21-55 as follows: for Group 5, the AM dose was ibrutinib, and the PM dose was vehicle only; for Group 7, ibrutinib was formulated in combination with belumosudil for the AM dose.

Rilzabrutinib. Rilzabrutinib was formulated with vehicle (0.5% MC). Rilzabrutinib was administered orally to mice of Groups 6 and 8 at a dose of 40 mg/kg. Animals were treated twice daily (BID) on Days 21-55.

Animals in Group 1 were not administered any treatments throughout the study. Animals in Groups 2-8 were administered vehicle or test article by oral gavage (PO) with dosing beginning on Day 21 and administered twice per day (BID), until Day 55. Animals in Groups 2, 3, and 6 received vehicle, or rilzabrutinib as indicated in Table 7 for both BID doses. Animals in Groups 4, 5 and 7 were administered ibrutinib and/or belumosudil for the AM dose and vehicle for the PM dose each day of dosing. Animals in Group 8 were administered rilzabrutinib and belumosudil for the AM dose and rilzabrutinib alone for the PM dose each day of dosing. On Day 33, the belumosudil dose for Groups 4, 7, and 8 was decreased from 125 mg/kg to 100 mg/kg,

### In-Life Monitoring and GVHD Assessment.

Animals were observed daily in order to assess possible differences among treatment groups and/or possible toxicity resulting from the treatments. Daily readouts were weight change, survival, standard GVHD score, modified GVHD score, incidence of diarrhea, and incidence of bloody stool. There were zero incidences of diarrhea and blood in stool throughout the study.

A clinical score for GVHD was obtained daily for the entire study duration as assessed by a standard scoring system (Table 4) and a modified scoring system for scGVHD (Table 5). The standard GVHD score was based on 5 criteria: percentage of weight change, posture (hunching), activity, fur texture, and skin integrity (maximum index=10). The modified GVHD score for was based on 5 criteria: percentage of weight change, posture (hunching), activity, fur texture, and skin integrity (maximum index=18). The overall score was reported, as well as the scores for each individual parameter.

On Days 33, 36, 39, 42 and 45, all animals were photographed under isoflurane anesthesia (topdown picture, plain white background) to assess disease severity. Each photo was tagged with the corresponding animal's number to aid identification.

### Supportive Care and Euthanasia Criteria.

Following TBI on Day −1 up to Day 8, all animals were given supplemental high-calorie highly palatable food in a dish on the bottom of the cage. Following transfer on Day 0, and then again as needed, all animals were given 1 mL/animal/day of supplemental fluids (warmed ringers solution) via subcutaneous injection. An additional 1 mL fluid may have been given in the afternoon on an as-needed basis. Any animal that exceeded 15% body weight loss were given highly palatable soft food and 1 mL supplemental fluids (warmed ringers solution) via subcutaneous injection once per day. Any animal that exceeded 20% body weight loss were given 1 mL supplemental fluids (warmed ringers solution) via subcutaneous injection twice per day. Animals that appeared to have been in pain were administered buprenorphine BID, as needed. Any animal that lost >30% of its body weight, were unable to right themselves, were cold to the touch, or was moribund was euthanized. Animals requiring euthanasia were euthanized by CO₂ inhalation and did not have organs collected.

### Sacrifice and Sample Collection.

On Day 56, all surviving animals were sacrificed by ketamine and xylazine overdose and organs detailed in Table 7 were collected. Blood, lungs, skin, and spleen were collected at sacrifice.

Blood. Prior to sacrifice, ~0.2 mL of blood was collected from all animals by retro-orbital bleed into K₂EDTA tubes. The blood was centrifuged, and the plasma collected and stored at −80° C.

Lung. The lungs were removed and whole lung and right lung were weighed. The right lung was tied off on the right bronchus, excised below the tie, snap frozen in liquid nitrogen and stored at −80° C. The left lung was insufflated with 10% Neutral Buffered Formalin (NBF), tied off on the trachea to maintain insufflation, fixed in 10% NBF for 24 hours, then moved to PBS.

Skin. Prior to collection, skin was shaved. A 1×1 cm portion of the skin (from the back of the animal, between the shoulders, closer to head) was excised, trimmed of any excess fat and/or connective tissue, sandwiched between foam in a tissue cassette, and placed in formalin. After 24 hours, the skin was transferred to PBS for storage until subsequent histological analyses. A second 1 cm×1 cm portion of the skin (from the back of the animal, between the shoulders, just below the first piece) was excised, trimmed of any excess fat and/or connective tissue, weighed, flash frozen, and stored at −80° C. If a visible lesion was present, it was collected in a way that captured part of it in both fixed and frozen sections.

Spleen. The spleen was excised and trimmed of any excess connective tissue. The spleen was then weighed, flash frozen, and stored at −80° C.

### Survival.

Animal deaths were evaluated during the course of the study. In this model, animal deaths are commonly attributable to severe disease. In this study, fifteen (15) animals were either found dead or were euthanized due to either euthanasia criteria or moribundity. Progression-free survival was defined as an increase in GVHD Score of less than or equal to 2 in comparison to Day 21 GVHD Score (standard or modified). A difference in GVHD score of 3 or higher relative to Day 21 meant that the animal no longer is in progression-free survival and had begun disease progression.

### Outcome Evaluation.

Study endpoints were body weight change, survival, progression-free survival, standard GVHD score, and modified scGVHD score.

### Statistical Analyses.

Data were evaluated using one-way ANOVA with Tukey's multiple comparison post-test to compare all groups. All statistical analyses were performed using GraphPad Prism 9.5.1 Software (La Jolla, CA). Statistical significance was achieved when p<0.05.

### Results.

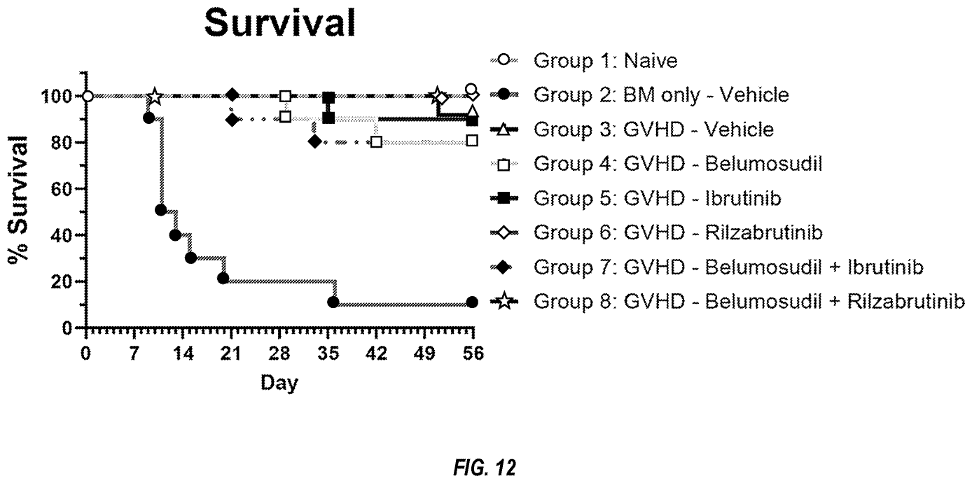

Survival. Survival was tracked for all animals during the course of the study and the percent survival is shown in FIG. 12.

All naïve animals (Group 1) survived to the conclusion of the study. Eight animals that received BM only (Group 2) died prior to the start of the study, and 10% survival was observed in this group by end of study. After the start of treatment on Day 21, all diseased animals treated with rilzabrutinib (Group 6) and belumosudil+rilzabrutinib (Group 8) survived to the conclusion of the study. In diseased animals, survival was 91.7% in vehicle-treated animals (Group 3), 90% with ibrutinib treatment (Group 5), 80% with belumosudil (Group 4), and 80% with belumosudil+ibrutinib (Group 7) treatment.

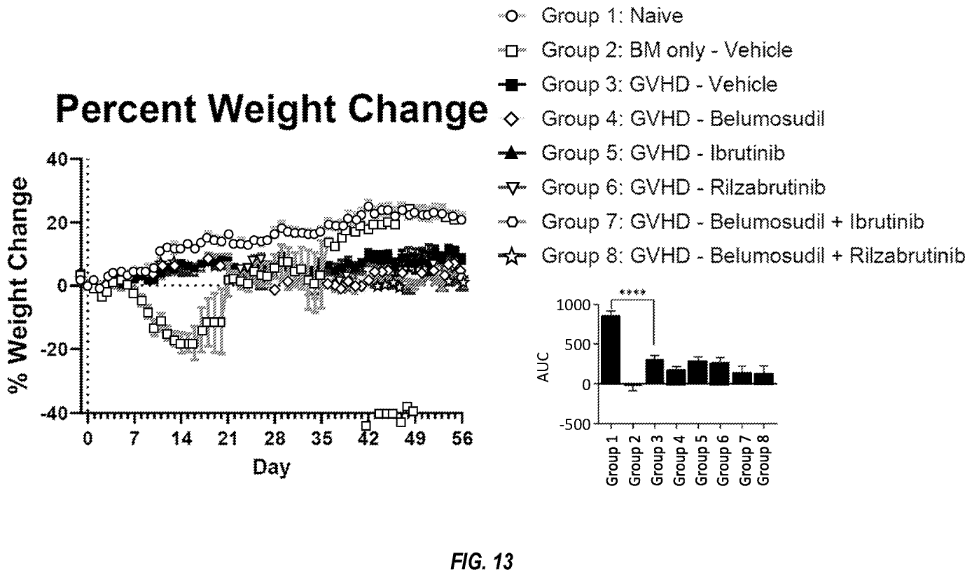

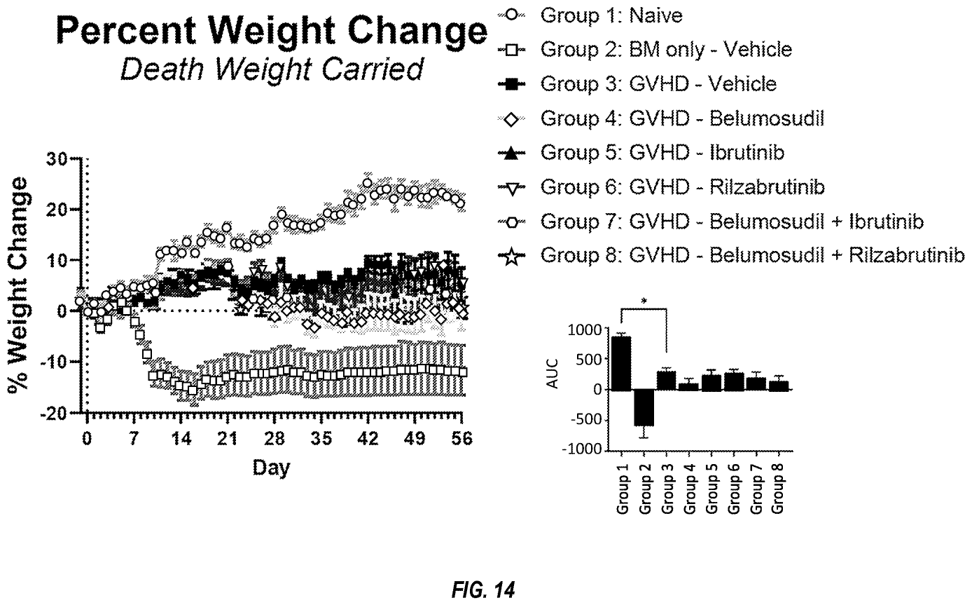

Body Weight. GVHD disease induction (cell transfer of splenocytes and bone marrow cells) prevented normal weight gain in all diseased animals. Survival past Day 14 indicates successful engraftment of donor cells. Percent weight change is plotted in FIG. 13 and percent weight change with death weight carried forward is plotted in FIG. 14.

Naïve animals (Group 1) displayed normal increases in weight throughout the study. By AUC analysis, naïve animals (Group 1) displayed significantly more weight gain (p<0.0001) compared to vehicle-treated diseased animals (Group 3). There were no significant differences in weight change observed between diseased animals treated with test articles (Groups 4-8) and vehicle-treatment (Group 3).

In order to account for changes in average weight due to deaths in the study which contribute to survivor bias, death weight was carried out until Day 56 and is presented in FIG. 14. By AUC analysis with death weight carried, naïve animals (Group 1) displayed significantly more weight gain (p<0.05 and p<0.001) compared to vehicle-treated diseased animals (Group 3).

After adjusting for survivor bias, BM only vehicle-treated animals (Group 2) displayed notably more body weight loss than all other groups, however, this observation was not statistically significant. There were no significant differences in weight change observed between diseased animals treated with test articles (Groups 4-8) and vehicle-treatment (Group 3).

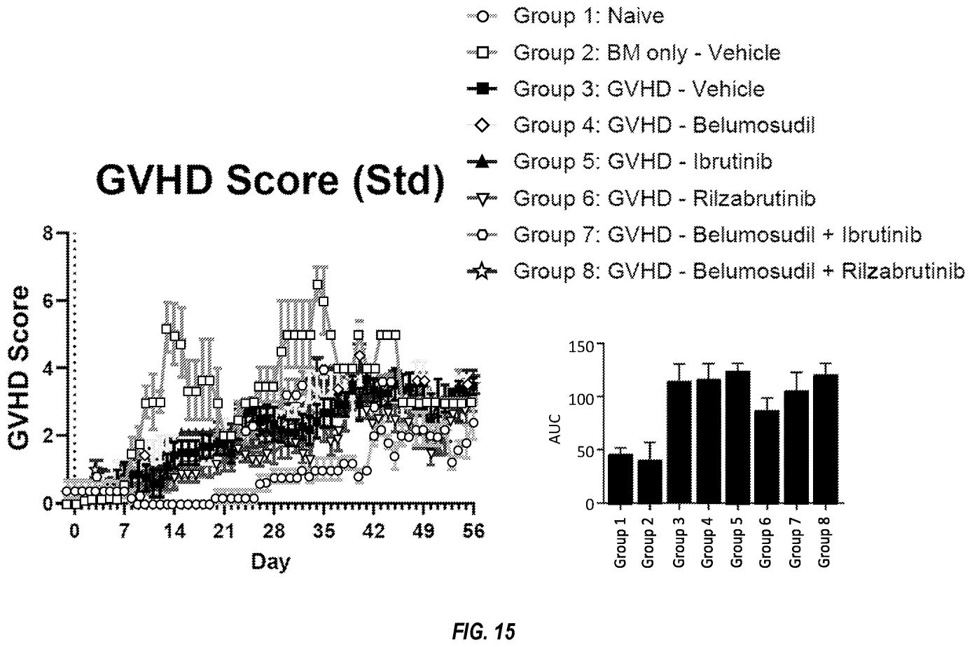

GVHD Score—Standard Scale. GVHD disease induction (cell transfer of splenocytes and bone marrow cells) was assessed using the multi-parameter GVHD scoring systems shown in Table 4. Survival of animals past Day 14 verifies successful engraftment of the transplanted cells. Standard GVHD scores are displayed in FIG. 15.

By AUC analysis from Day 0, vehicle-treated diseased animals (Group 3) displayed significantly higher standard GVHD scores (p<0.05) compared to naïve animals (Group 1). There were no significant differences in standard GVHD scores observed between diseased animals treated with test articles (Groups 4-8) and vehicle-treatment (Group 3). However, diseased animals treated with rilzabrutinib (Group 6) had mildly lower standard GVHD scores than vehicle treatment (Group 3).

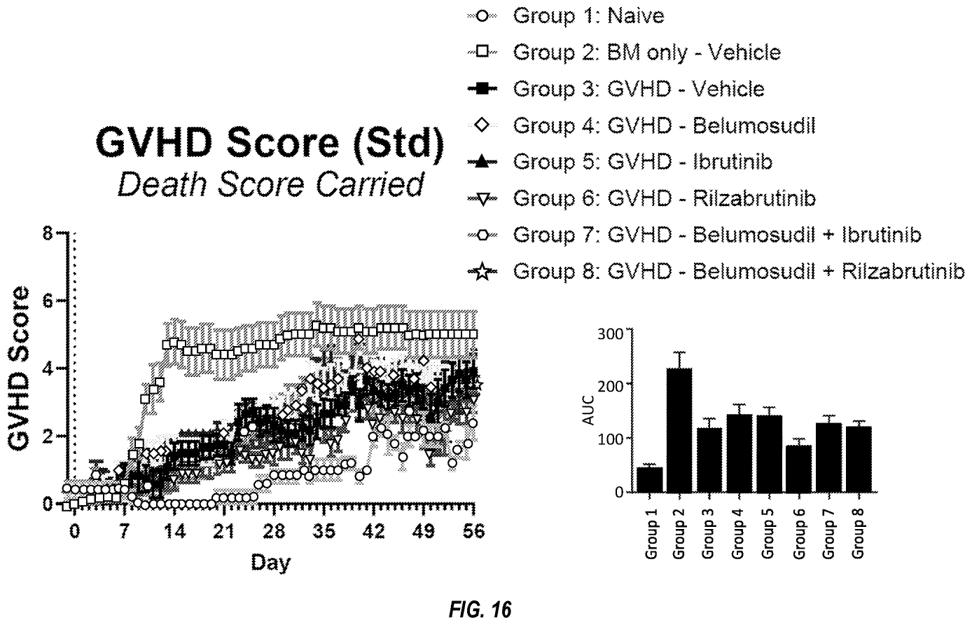

In order to account for changes in GVHD score due to deaths in the study which contribute to survivor bias, death score was carried out until Day 56 and is presented in FIG. 16. By AUC analysis with death score carried, BM only animals (Group 2) had notably higher standard GVHD scores relative to all groups, however, this was not statistically significant. The trend of higher standard GVHD scores in vehicle-treated diseased animals (Group 3) compared to naïve animals (Group 1) was dampened after adjusting for survivor bias and was no longer statistically significant. There were no significant differences in standard GVHD scores observed between diseased animals treated with test articles (Groups 4-8) and vehicle-treatment (Group 3). However, the trend of lower standard GVHD scores with rilzabrutinib (Group 6) compared to vehicle treatment (Group 3) remained.

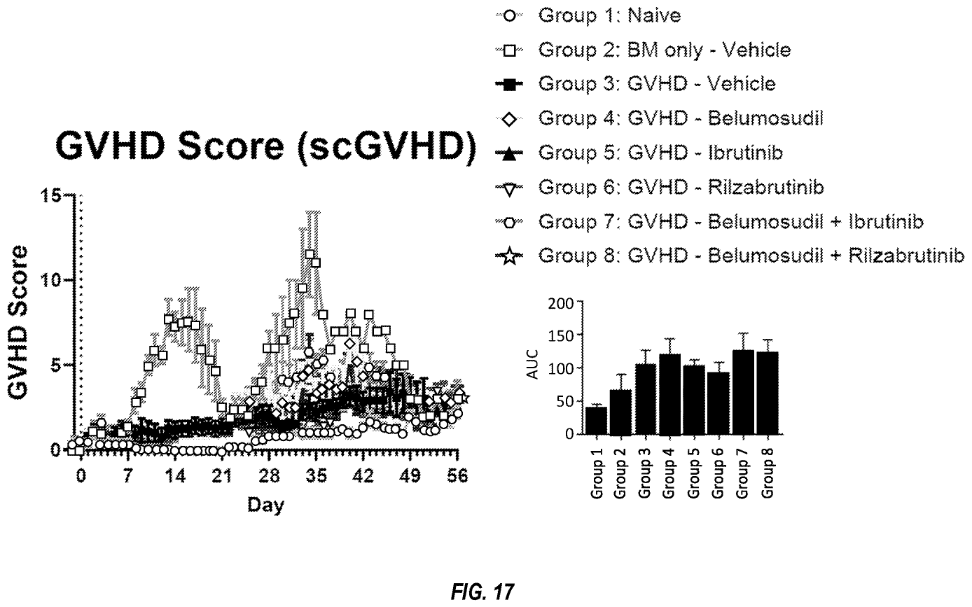

GVHD Score—Modified Scale. GVHD disease induction (cell transfer of splenocytes and bone marrow cells) was assessed using the modified multi-parameter GVHD scoring system shown in Table 5, for sclerodermatous GVHD. Survival of animals past Day 14 verifies successful engraftment of the transplanted cells. Modified GVHD scores are displayed in FIG. 17.

By AUC analysis, there were no statistically significant differences in scGVHD scores observed between the groups. However, the following trends were observed. Diseased animals treated with vehicle (Group 3) demonstrated notably higher scGVHD scores than naïve animals (Group 1). BM only animals (Group 2) displayed mildly lower modified GVHD scores compared to diseased animals treated with vehicle (Group 3).

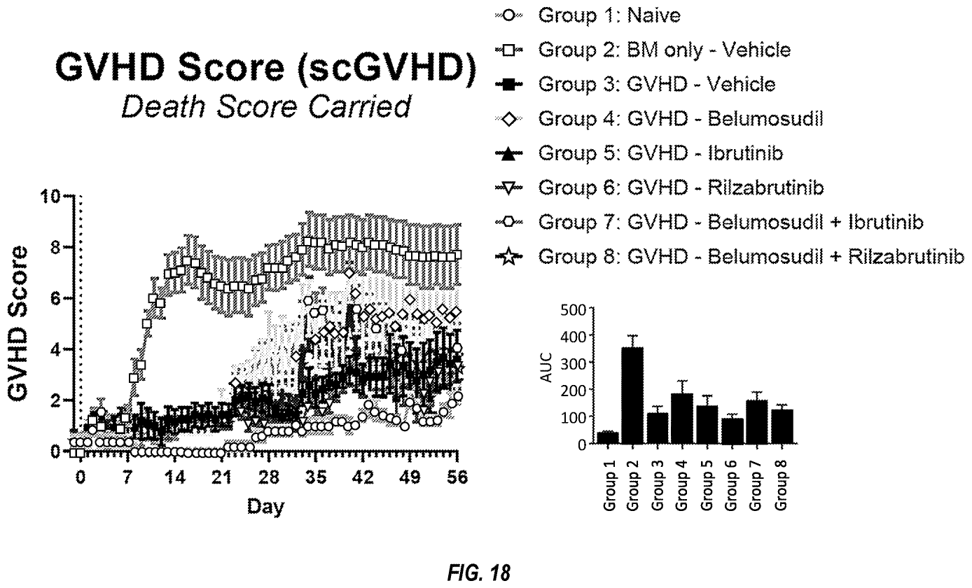

In order to account for changes in average scGVHD score due to deaths in the study which contribute to survivor bias, death score was carried out until Day 56 and is presented in FIG. 18. By AUC analysis, with death score carried, there were no statistically significant differences in scGVHD scores observed between the groups. However, diseased animals treated with vehicle (Group 3) continued to demonstrate notably higher scGVHD scores than naïve animals (Group 1). BM only animals (Group 2) displayed notably higher modified GVHD scores compared to diseased animals treated with vehicle (Group 3). There were no trends of improved scGVHD scores observed with test article treatment compared to vehicle-treatment.

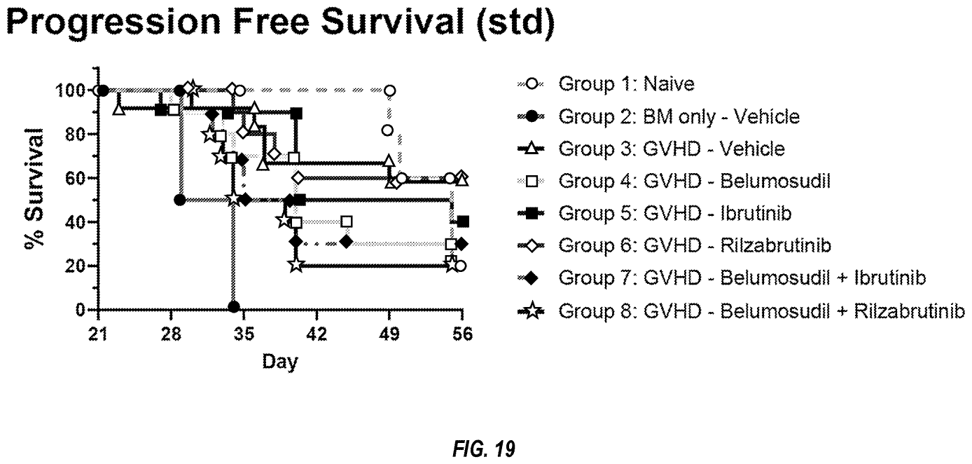

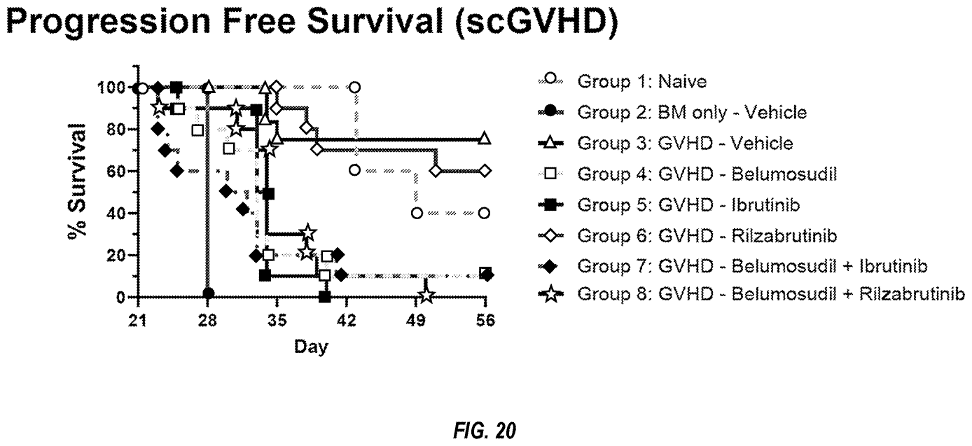

Progression-Free Survival. Progression free survival (PFS) was tracked for all animals during the course of the study and the percent progression free survival was plotted, as shown in FIG. 19 (standard GVHD scale) and FIG. 20 (modified scGVHD scale). Progression free survival was defined as an increase in GVHD Score of less than or equal to 2 in comparison to Day 21 GVHD Score (standard or modified). Disease progression was defined as an increase in GVHD Score of greater than 2 in comparison to Day 21 GVHD Score (standard or modified).

The progression free survival analysis by the standard GVHD scale showed that ~80% of the naïve animals (Group 1) had changes in scores greater than 2. By the scGVHD scale, ~60% of the naïve animals (Group 1) had changes in scores greater than 2. However, this was largely due to barbering that occurred in the naïve group, which led to mild hair loss and contributed to higher-than-expected fur texture scores.

Progression free survival analysis using the standard GVHD scoring scale showed that ~58% of diseased animals treated with vehicle (Group 3) did not display progressive disease by Day 56. Analysis using the modified scGVHD scale showed that ~75% of vehicle-treated diseased animals (Group 3) did not display progressive disease by Day 56. These results correlate with a lower disease severity and disease penetrance in this study.

### Conclusions.

All naïve animals as well as diseased animals treated with rilzabrutinib, and combination belumosudil+rilzabrutinib survived to the conclusion of the study. A 10% survival was observed in animals that received BM only. In diseased animals, survival was 91.7% in vehicle-treated animals, 90% with ibrutinib treatment, and 80% with belumosudil and 80% with belumosudil+ibrutinib treatment.

By AUC analysis, naïve animals displayed significantly more weight gain compared to vehicle-treated diseased animals. After adjusting for survivor bias with death weight carried, naïve animals continued to display significantly more weight gain compared to vehicle-treated diseased animals. BM only vehicle-treated animals displayed notably more body weight loss than all other groups, however, this observation was not statistically significant. There were no significant differences in weight change observed between diseased animals treated with test articles and vehicle-treatment.

By AUC analysis, vehicle-treated diseased animals displayed significantly higher standard GVHD scores compared to naïve animals. There were no significant differences in standard GVHD scores observed between diseased animals treated with test articles and vehicle. However, diseased animals treated rilzabrutinib had mildly lower standard GVHD scores than vehicle treatment. With death score carried, BM only animals had notably higher standard GVHD scores relative to all groups, however this finding was not statistically significant. The trend of higher standard GVHD scores in vehicle-treated diseased animals compared to naïve animals was dampened after adjusting for survivor bias and was no longer statistically significant. The trend of lower standard GVHD scores with rilzabrutinib compared to vehicle treatment remained when death scores were carried.

By AUC analysis, there were no statistically significant differences in scGVHD scores observed between the groups. However, diseased animals treated with vehicle demonstrated notably higher scGVHD scores than naïve animals. BM only animals displayed mildly lower modified GVHD scores compared to diseased animals treated with vehicle. With death score carried, diseased animals treated with vehicle continued to demonstrate notably higher scGVHD scores than naïve animals. Although not statistically significant, notably higher scGVHD scores were observed in animals that received BM only compared to all other groups when death score was carried. There were no trends of improved scGVHD scores observed with test article treatment compared to vehicle-treatment.

Progression free survival analysis using the standard GVHD scoring scale showed that ~58% of diseased animals treated with vehicle did not display progressive disease by Day 56.

Analysis using the modified scGVHD scale showed that ~75% of vehicle-treated diseased animals did not display progressive disease by Day 56. These results correlate with a lower disease severity and disease penetrance in this study.

As indicated by the data, there are limitations associated with this study. For example, the disease induction was not robust. Also, the positive control groups did not show a statistically significant difference. Due to such limitations, the study outcome is inconclusive.

Although the foregoing invention has been described in some detail by way of illustration and example for purposes of clarity of understanding, the descriptions and examples should not be construed as limiting the scope of the invention. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference. To the extent any material incorporated herein by reference is inconsistent with the express content of this disclosure, the express content controls.

What is claimed is:

1. A method of treating a disease or disorder selected from systemic sclerosis and a transplant-associated dysfunction in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

2. A method of treating a disease or disorder selected from graft-versus-host disease (GVHD), systemic sclerosis (scleroderma), chronic lung allograft dysfunction (CLAD), restrictive allograft syndrome (RAS), and bronchiolitis obliterans syndrome (BOS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

3. A method for treating graft-versus-host disease (GVHD) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

4. A method for treating systemic sclerosis (scleroderma) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

5. A method for treating chronic lung allograft dysfunction (CLAD) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

6. A method for treating restrictive allograft syndrome (RAS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}j-N-(propan-2-yl) acetamide or a pharmaceutically acceptable salt thereof.

7. A method for treating bronchiolitis obliterans syndrome (BOS) in a human patient in need thereof comprising administering to the human patient a therapeutically effective amount of a combination comprising: (a) a Bruton's tyrosine kinase (BTK) inhibitor, and (b) 2-{3-[4-(1H-indazol-5-ylamino)-2-quinazolinyl]phenoxy}-N-(propan-2-yl) acetamide or a pharmaceutically acceptable salt thereof.

8. The method of any one of claims 1-7, wherein the BTK inhibitor is a reversible BTK inhibitor.

9. The method of any one of claims 1-7, wherein the BTK inhibitor is an irreversible BTK inhibitor.

10. The method of any one of claims 1-9, wherein the BTK inhibitor is (i) (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof; (ii) 1-[(3R)-3-[4-amino-3-(4-phenoxyphenyl)-1H-pyrazolo[3,4-d]pyrimidin-1-yl]-1-piperidinyl]-2-propen-1-one or a pharmaceutically acceptable salt thereof; or (iii) (4-amino-3-(4-phenoxyphenyl)-1-[(3R)-1-(prop-2-enoyl)piperidin-3-yl]-1,3-dihydro-2H-imidazo[4,5-c]pyridin-2-one) or a pharmaceutically acceptable salt thereof.

11. The method of claim 10, wherein the BTK inhibitor is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

12. A method for treating graft-versus-host disease (GVHD) in a human patient in need thereof comprising administering to the human patient a therapeutically effective amount of a combination comprising: (a) (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof, and (b) 2-{3-[4-(11H-indazol-5-ylamino)-2-quinazolinyl]phenoxy}-N-(propan-2-yl) acetamide or a pharmaceutically acceptable salt thereof.

13. The method of any one of claims 2, 3, and 12, wherein GVHD is chronic GVHD (cGVHD).

14. The method of any one of claims 1-13, wherein 2-{3-[4-(1H-indazol-5-ylamino)-2-quinazolinyl]phenoxy}-Nd-(propan-2-yl) acetamide or a pharmaceutically acceptable salt thereof is administered to the human patient at a daily dosage of up to about 400 mg.

15. The method of claim 14, wherein 2-{3-[4-(1H-indazol-5-ylamino)-2-quinazolinyl]phenoxy}-N-(propan-2-yl) acetamide, or a pharmaceutically acceptable salt thereof, is administered to the human patient at a daily dosage of about 50-400 mg.

16. The method of claim 14 or 15, wherein 2-{3-[4-(l H-indazol-5-ylamino)-2-quinazolinyl]phenoxyl}-N-(propan-2-yl) acetamide, or a pharmaceutically acceptable salt thereof, is administered to the human patient at a dose of about 50 mg, 100 mg, 150 mg, or 200 mg.

17. The method of claim 16, wherein the dose is administered to the human patient once or twice daily.

18. The method of any one of claims 1-17, wherein 2-{3-[4-(1H-indazol-5-ylamino)-2-quinazolinyl]phenoxy}-N-(propan-2-yl) acetamide, or a pharmaceutically acceptable salt thereof, is administered orally.

19. The method of any one of claims 10-18, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a daily dosage of up to about 800 mg.

20. The method of claim 19, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a daily dosage of about 50-800 mg.

21. The method of claim 19 or 20, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient at a dose of about 100 mg, 200 mg, or 400 mg.

22. The method of claim 21, wherein the dose of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof is administered to the human patient once a day or twice a day.

23. The method of any one of claims 10-22, wherein the (E) isomer (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

24. The method of any one of claims 10-22, wherein the (Z) isomer (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

25. The method of any one of claims 10-22, wherein a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered to the human patient.

26. The method of any one of claims 10-25, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, is administered orally.

27. The method of any one of claims 10-26, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered separately.

28. The method of claim 27, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered sequentially.

29. The method of any one of claims 10-28, wherein (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, or a pharmaceutically acceptable salt thereof, and 2-{3-[4-(1H-indazol-5-ylamino)-2-quinazolinyl]phenoxy}-N-(propan-2-yl) acetamide, or a pharmaceutically acceptable salt thereof, are administered simultaneously.
